(19) United States
(12) Patent Application Publication
(10) Pub. No.: US 2025/0339121 A1
(43) Pub. Date: Nov. 6, 2025

(54) SYSTEM AND METHOD FOR MONITORING AUTOREGULATION

(71) Applicant: Becton, Dickinson and Company, Franklin Lakes, NJ (US)

(72) Inventors: Blake W. Axelrod, South Pasadena, CA (US); Antonio Albanese, Mission Viejo, CA (US); Paul B. Benni, Action, MA (US); Jacobus Jozef Gerardus Maria Settels, Amsterdam (NL)

(21) Appl. No.: 19/265,932

(22) Filed: Jul. 10, 2025

Related U.S. Application Data
(63) Continuation of application No. PCT/US2024/011063, filed on Jan. 10, 2024.
(60) Provisional application No. 63/479,264, filed on Jan. 10, 2023.

(51) Int. Cl. A61B 8/06; A61B 8/00

(52) U.S. Cl. CPC A61B 8/06; A61B 8/461; A61B 8/488

(57) ABSTRACT

A system includes a first sensor to continuously measure a signal of a renal blood flow of a patient. A second sensor continuously measures an arterial pressure signal of the patient. A blood flow monitor is in communication with the first and second sensors. The blood flow monitor includes system memory that stores monitoring software code and a processor. The processor is configured to execute the monitoring software code to estimate a flow rate of the renal blood flow of the patient from the signal of the renal blood flow and monitor changes in the flow rate of the renal blood flow over time. The processor is also configured to execute the monitoring software code to monitor changes in the arterial pressure signal over time and evaluate a mathematical relationship between the changes in the arterial pressure signal and the changes in the flow rate of the renal blood flow.

### CROSS-REFERENCE TO RELATED APPLICATION(S)

This application is a continuation of International Application No. PCT/US2024/011063, filed Jan. 10, 2024, entitled “SYSTEM AND METHOD FOR MONITORING AUTOREGULATION,” which claims the benefit of U.S. Provisional Application No. 63/479,264, filed Jan. 10, 2023, and entitled “SYSTEM AND METHOD FOR MONITORING AUTOREGULATION,” the disclosures of which are hereby incorporated by reference in their entireties.

### BACKGROUND

This present disclosure relates to monitoring perfusion and blood flow in organs in patients and, more specifically, to medical apparatuses and methods for measuring and/or monitoring autoregulation.

Autoregulation is the ability of an organ to regulate the flow of blood locally through the organ. The failure or loss of autoregulation is a risk factor for organ damage and often a sign of breakdown of the compensatory circulatory processes of the patient's body. Different organs display varying degrees of autoregulatory behavior. The kidney and the brain are two high blood flow organs and the two most tightly autoregulated organs in the human body. The goals of adequate autoregulation between the brain and the kidneys are very different. The goal of cerebral autoregulation is to maintain sufficient oxygen to the brain. The goal of renal autoregulation is to achieve adequate tubular and glomerular flow.

Myogenic response and Tubular Glomerular Feedback (TGF) response are two mechanisms that dominate renal autoregulation. The myogenic response occurs in the afferent arterioles and is a fast and ballistic response to mitigate systole and similar surges in blood pressure. The myogenic response is triggered by hoop stress in the afferent arterioles, which is a purely mechanical and protective response. The TGF response is a slow, closed loop response that modulates renal blood flow in response to salt concentrations in the distal tubules. In animal studies the lower limits of renal autoregulation are much higher than cerebral autoregulation: 70 mmHg vs 30 mmHg.

A plurality of factors (e.g., a hardening of the arteries that occurs with advancing age) can change the characteristics of a vascular reactivity response, and these factors can in turn change relevant autoregulation characteristics of the patient. Hence, the autoregulation range of blood flow due to changing blood pressure can vary between patients and within patients and cannot be assumed to be a constant. Methods and apparatus for determining whether a particular patient's autoregulation is functioning, and the potential range to manage blood pressure variability, would be a great help to a clinician. What is needed is an apparatus and method for monitoring autoregulation that is an improvement over those known in the prior art, including one that identifies and accounts for factors that may confound an autoregulation determination or measurement.

### SUMMARY

A method for continuously monitoring a kidney of a patient during a surgery, a medical procedure, or a medical observation includes continuously measuring a signal of a renal blood flow of the patient with a first sensor attached to the patient. The first sensor is in communication with a blood flow monitor. A processor of the blood flow monitor estimates a flow rate of the renal blood flow of the patient from the signal of the renal blood flow. The processor also monitors changes in the flow rate of the renal blood flow over time. An arterial pressure signal of the patient is continuously measured by a second sensor. The second sensor is in communication with the blood flow monitor. The processor also monitors changes in the arterial pressure signal over time and evaluates a mathematical relationship between the changes in the arterial pressure signal and the changes in the flow rate of the renal blood flow.

A system includes a first sensor configured to continuously measure a signal of a renal blood flow of a patient during a surgery, a medical procedure, or a medical observation. A second sensor is configured to continuously measure an arterial pressure signal of the patient during the surgery, the medical procedure, or the medical observation. A blood flow monitor is in communication with the first sensor and the second sensor. The blood flow monitor includes a system memory that stores monitoring software code and a processor. The processor is configured to execute the monitoring software code to estimate a flow rate of the renal blood flow of the patient from the signal of the renal blood flow and monitor changes in the flow rate of the renal blood flow over time. The processor is also configured to execute the monitoring software code to monitor changes in the arterial pressure signal over time and evaluate a mathematical relationship between the changes in the arterial pressure signal and the changes in the flow rate of the renal blood flow.

A method for continuously monitoring a kidney of a patient during a surgery, a medical procedure, or a medical observation, includes continuously measuring a Doppler flow signal of a renal blood flow of the patient with an ultrasound transducer probe. The ultrasound transducer probe is attached in a stationary position to an abdomen of the patient and is in communication with a processor of a blood flow monitor. The processor monitors changes in the renal blood flow over time. A hemodynamic pressure sensor continuously measures an arterial pressure signal of the patient. The hemodynamic pressure sensor is in communication with the blood flow monitor. The processor monitors changes in the arterial pressure signal over time and evaluates a mathematical relationship between the changes in the arterial pressure signal and the changes in the renal blood flow. The processor determines an autoregulation profile of the renal blood flow of the patient based on the mathematical relationship between the changes in the arterial pressure signal and the changes in the renal blood flow.

A system includes an ultrasound transducer probe with a two-dimensional array of transducer elements configured to continuously measure a Doppler flow signal of a renal blood flow of a patient during a surgery, a medical procedure, or a medical observation. An adhesive patch is connected to the ultrasound transducer probe and is configured to attach the ultrasound transducer probe to the patient and maintain contact between the patient and the ultrasound transducer probe without an operator. The system further includes a hemodynamic pressure sensor configured to continuously measure an arterial pressure signal of the patient during the surgery, the medical procedure, or the medical observation. A blood flow monitor is in communication with the ultrasound transducer probe and the hemodynamic pressure sensor. The blood flow monitor includes a system memory that stores monitoring software code and a processor. The processor is configured to execute the monitoring software code to determine changes in the renal blood flow of the patient from the Doppler flow signal of the renal blood flow and to monitor the changes in the renal blood flow over time. The processor is also configured to execute the monitoring software code to monitor changes in the arterial pressure signal over time and evaluate a mathematical relationship between the changes in the arterial pressure signal and the changes in the renal blood flow.

### BRIEF DESCRIPTION OF THE DRAWINGS

FIG. 1 is a schematic diagram illustrating an example monitoring system with a blood flow monitor, an ultrasound transducer probe attached to an abdomen of a patient by an adhesive patch, and a hemodynamic pressure sensor connected to the patient for sensing hemodynamic data representative of an arterial pressure of the patient.

FIG. 2 is another schematic diagram illustrating the blood flow monitor of FIG. 1 connected to an ultrasound transducer probe with a two-dimensional array of transducer elements.

FIG. 3 is a schematic diagram of an ultrasound transducer probe attached to an abdomen of a patient by an adhesive patch to monitor a renal flow of a kidney of the patient.

FIG. 4A is another schematic diagram of an ultrasound transducer probe attached to an abdomen of a patient by an adhesive patch to monitor a kidney of the patient.

FIG. 4B is another schematic diagram of an ultrasound transducer probe attached to an abdomen of a patient by an adhesive patch to monitor a kidney of the patient.

FIG. 5 is a perspective view of an example minimally invasive hemodynamic pressure sensor for sensing hemodynamic data representative of arterial pressure of a patient.

FIG. 6 is a perspective view of an example non-invasive hemodynamic pressure sensor for sensing hemodynamic data representative of arterial pressure of a patient.

FIG. 7 is a diagrammatic representation of a time domain method for determining a mathematical relationship between a mean arterial pressure (MAP) of a patient and a renal blood flow of a kidney of the patient.

FIG. 8 is a diagrammatic representation of a frequency domain method for determining a mathematical relationship between a mean arterial pressure (MAP) of a patient and a renal blood flow of a kidney of the patient.

FIG. 9 is a chart of correlation versus MAP of a patient.

FIG. 10 is a plot of correlation between changes in a flow rate of a renal blood flow of a patient and changes in a MAP versus the MAP of the patient.

FIG. 11 is a block diagram of a method for determining an autoregulation profile of a renal flow of a patient.

FIG. 12 is a chart from an experiment demonstrating the monitoring system of FIG. 1.

FIG. 13 is a plot of a renal blood flow versus a MAP of a test subject from the experiment of FIG. 12.

FIG. 14 is a chart of correlation versus MAP of the test subject from the experiment of FIG. 12.

FIG. 15 is a block diagram of a method for continuously monitoring an autoregulation profile of a renal blood flow of a patient during a surgery, medical procedure, or medical observation with a blood flow monitor for risk of acute kidney injury.

### DETAILED DESCRIPTION

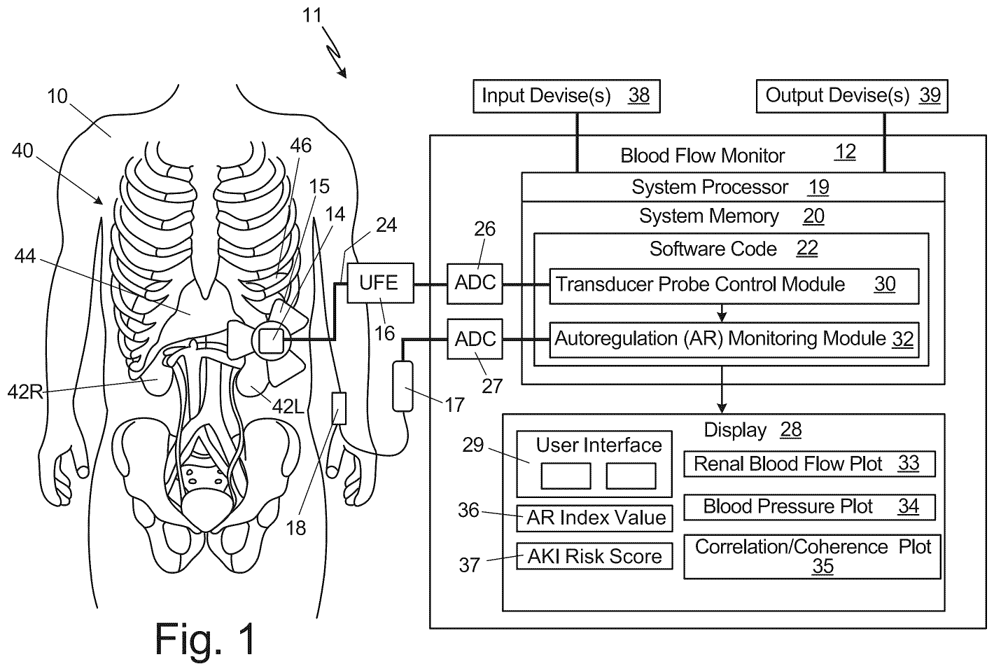

The present disclosure is directed to a monitoring system and a method to monitor in real time a blood flow of an abdominal organ, such as a kidney, of a patient during a surgery, medical procedure, or medical observation. The monitoring system includes a blood flow monitor, an ultrasound transducer probe, and a hemodynamic pressure sensor. The monitoring system also includes an adhesive patch that can attach the ultrasound transducer probe to the patient and keep the ultrasound transducer probe attached to the patient through the surgery, the medical procedure, or the medical observation of the patient without assistance from an ultrasound operator. The blood flow monitor determines an autoregulation index of a renal blood flow of the kidney of the patient based on information received by the blood flow monitor from the ultrasound transducer probe and the hemodynamic pressure sensor. The index is determined as a function of time, and as a function of blood pressure. The blood flow monitor determines an autoregulation profile of a renal blood flow of the kidney of the patient based on autoregulation index information received by the blood flow monitor from the ultrasound transducer probe and the hemodynamic pressure sensor. The autoregulation index and the profile of the renal blood flow autoregulation of the patient can be continuously updated and outputted to a display during the surgery, medical procedure, or medical observation so that medical personnel can be informed in real time of the autoregulation profile of the renal blood flow of the patient. The monitoring system is described in detail below with reference to FIGS. 1-15.

FIG. 1 is a schematic diagram of patient 10 and monitoring system 11 that continuously monitors an organ blood flow of patient 10 during a surgery, medical procedure, or medical observation. As shown in the example of FIG. 1, monitoring system 11 can include blood flow monitor 12, ultrasound transducer probe 14, adhesive patch 15, ultrasound front-end (UFE) circuitry 16, hemodynamic pressure sensor 17, radial arterial catheter 18, system processor 19, system memory 20 with software code 22, probe cable(s) 24, first analog-to-digital (ADC) converter 26, second analog-to-digital (ADC) converter 27, and display 28. Software code 22 can include transducer probe control module 30 and autoregulation (AR) monitoring module 32. Display 28 can include user interface 29, first plot 33, second plot 34, third plot 35, autoregulation index value 36, and injury score indicator 37. Monitoring system 11 can also include input device(s) 38 and output device(s) 39. FIG. 1 also shows abdomen 40 of patient 10 along with kidneys 42L and 42R, liver 44, and spleen 46. In the example of FIG. 1, monitoring system 11 is monitoring a renal blood flow of kidney 42L of patient 10. In other examples, monitoring system 11 can be used to monitor hepatic blood flow of liver 44, to monitor celiac blood flow of spleen 46, the pancreas (not shown), and the stomach (not shown) of patient 10, and/or to monitor portal blood flow from the stomach of patient 10. Thus, blood flow monitor 12 can be adapted as an organ blood flow monitor 12 for any organ of patient 10.

Blood flow monitor 12, can be, e.g., an integrated hardware unit that includes system processor 19, system memory 20, display 28, UFE circuitry 16, first ADC 26, and second ADC 27. In other examples, any one or more components and/or described functionality of organ blood flow monitor can be distributed among multiple hardware units. For instance, in some examples, display 28 can be a separate display device that is remote from blood flow monitor 12 and operatively coupled with blood flow monitor 12 as an output device 39. In general, though illustrated and described in the example of FIG. 1 as an integrated hardware unit, it should be understood that blood flow monitor 12 can include any combination of devices and components that are electrically, communicatively, or otherwise operatively connected to perform functionality attributed herein to blood flow monitor 12. Input device(s) 38 can be connected to blood flow monitor 12 such that a user may input data and/or commands into blood flow monitor 12. Non-limiting examples of input device(s) 38 includes a keyboard, a touchpad, and/or other devices whereby a user may input data and/or commands into blood flow monitor 12. Input device(s) 38 can also include a port configured for communication with an external input device via hardwire or wireless connection.

Ultrasound transducer probe 14 is a first sensor of monitoring system 11. Ultrasound transducer probe 14 can be attached or secured to patient 10 by adhesive patch 15. In the example of FIG. 1, ultrasound transducer probe 14 is positioned on abdomen 40 of patient 10 over at least a portion of kidney 42L. Adhesive patch 15 can include a sheet of structural material, such as fabric or flexible plastic, with a layer of bonding adhesive deposited on a face of the sheet. Adhesive patch 15 can be bonded to or mechanically connected to ultrasound transducer probe 14, or to a frame (not shown) connected to a base of ultrasound transducer probe 14, and can extend outward from ultrasound transducer probe 14 along a surface of abdomen 40 of patient 10. In other examples, adhesive patch 15 can be placed over ultrasound transducer probe 14 to attach ultrasound transducer probe 14 to abdomen 40 of patient 10. Adhesive patch 15 keeps ultrasound transducer probe 14 attached to patient 10 and secured in place throughout a duration of the surgery, medical procedure, or medical observation of patient 10. Since adhesive patch 15 keeps ultrasound transducer probe 14 immobile and in contact with patient 10, an ultrasound operator or technician is not needed during the surgery, medical procedure, or medical observation to keep ultrasound transducer probe 14 in position. A coupling layer (not shown) with a couplant material can be positioned between a skin of patient 10 and ultrasound transducer probe 14. The coupling layer enables ultrasonic energy transmission between the skin of patient 10 and ultrasound transducer probe 14.

In the example of FIG. 1, the ultrasound transducer probe 14 detects and continuously senses a Doppler flow signal of the renal blood flow of kidney 42L during the surgery, the medical procedure, or the medical observation of patient 10. The term “continuously” as used herein means that ultrasound transducer probe 14 senses the Doppler flow signal of the renal blood flow of kidney 42L and collects patient data on a periodic basis during the monitoring time period, which periodic basis is sufficiently frequent that the periodic basis may be considered to be clinically continuous. For example, ultrasound transducer probe 14 can sample the Doppler flow signal of the renal blood flow of kidney 42L every ten seconds or less (<10 seconds), and can be configured to sample data more frequently (e.g., every two seconds or less). The present disclosure is not limited to any particular device settings or sampling rate.

Ultrasound transducer probe 14 can be operatively connected to blood flow monitor 12 by cable(s) 24. Via cable(s) 24, ultrasound transducer probe 14 can receive electrical signals from the UFE circuitry 16 of the blood flow monitor 12 and can relay the received ultrasound signals from patient 10 to blood flow monitor 12 for extraction of the Doppler flow signal of the renal blood flow of kidney 42L. In other examples, UFE circuitry 16 is combined with ultrasound transducer probe 14, can be battery powered and can include a receiver to wirelessly receive commands from blood flow monitor 12. The combined UFE circuitry 16 and ultrasound transducer probe 14 can also include a transmitter to wirelessly communicate the Doppler flow signal of the renal blood flow of kidney 42L to blood flow monitor 12 for analysis. In some examples, the combined ultrasound transducer probe 14 and UFE circuitry 16 provide the Doppler flow signal to blood flow monitor 12 as an analog signal, which is converted by first ADC 26 to digital hemodynamic data representative of the renal blood flow of kidney 42L. In other examples, the combined ultrasound transducer probe 14 and UFE circuitry 16 can provide the sensed Doppler flow signal to blood flow monitor 12 in digital form, in which case blood flow monitor 12 may not include or utilize first ADC 26. In yet other examples, ultrasound transducer probe 14 can provide the Doppler flow signal of the renal blood flow of kidney 42L to blood flow monitor 12 as an analog signal, which is analyzed in its analog form by blood flow monitor 12.

Hemodynamic pressure sensor 17 is a second sensor of monitoring system 11. In the example of FIG. 1, hemodynamic pressure sensor 17 is a minimally invasive hemodynamic pressure sensor attached to patient 10 via radial arterial catheter 18 inserted into an arm of patient 10. In other examples, hemodynamic pressure sensor 17 can be attached to patient 10 via a femoral arterial catheter inserted into a leg of patient 10, or hemodynamic pressure sensor 17 can be placed non-invasively on an extremity of patient 10, such as a wrist, an arm, a finger, an ankle, a toc, or other extremity of patient 10. Hemodynamic pressure sensor 17 continuously senses hemodynamic data representative of an arterial pressure of patient 10 during the surgery, the medical procedure, or the medical observation of patient 10. The term “continuously” as used herein means that hemodynamic pressure sensor 17 senses and collects patient data on a periodic basis during the monitoring time period, which periodic basis is sufficiently frequent that the periodic basis may be considered to be clinically continuous. For example, hemodynamic pressure sensor 17 can sample a waveform of the hemodynamic data representative of the arterial pressure of patient 10 at a rate of at least 10 Hz, at least 20 Hz, at least 60 Hz, at least 100 Hz, or at least 200 Hz. In other examples, hemodynamic pressure sensor 17 can sample an average of the signal of the hemodynamic data representative of the arterial pressure of patient 10 over a window of time, such as every ten seconds or less (<10 seconds). Hemodynamic pressure sensor 17 can sample an average of the signal of the hemodynamic data representative of the arterial pressure of patient 10 more frequently, such as every two seconds or less. In other examples, can sample an average of the signal of the hemodynamic data representative of the arterial pressure of patient 10 over a rolling window of time. The present disclosure is not limited to any particular device settings or sampling rate.

Hemodynamic pressure sensor 17 is operatively connected to blood flow monitor 12 (e.g., electrically and/or communicatively connected via wired or wireless connection, or both) to provide the sensed hemodynamic data to blood flow monitor 12. In some examples, hemodynamic pressure sensor 17 provides the sensed hemodynamic data representative of the arterial pressure of patient 10 to blood flow monitor 12 as an analog signal, which is converted by second ADC 27 to digital hemodynamic data representative of the arterial pressure of patient 10. In other examples, hemodynamic pressure sensor 17 can provide the sensed hemodynamic data representative of the arterial pressure of patient 10 to blood flow monitor 12 in digital form, in which case blood flow monitor 12 may not include or utilize second ADC 27. In yet other examples, hemodynamic pressure sensor 17 can provide the hemodynamic data representative of the arterial pressure of patient 10 to blood flow monitor 12 as an analog signal, which is analyzed in its analog form by blood flow monitor 12.

System memory 20 can be configured to store information within blood flow monitor 12 during operation. System memory 20, in some examples, is described as computer-readable storage media. In some examples, a computer-readable storage medium can include a non-transitory medium. The term “non-transitory” can indicate that the storage medium is not embodied in a carrier wave or a propagated signal. In certain examples, a non-transitory storage medium can store data that can, over time, change (e.g., in RAM or cache). System memory 20 can include volatile and non-volatile computer-readable memories. Examples of volatile memories can include random access memories (RAM), dynamic random access memories (DRAM), static random access memories (SRAM), and other forms of volatile memories. Examples of non-volatile memories can include, e.g., magnetic hard discs, optical discs, flash memories, or forms of electrically programmable memories (EPROM) or electrically erasable and programmable (EEPROM) memories.

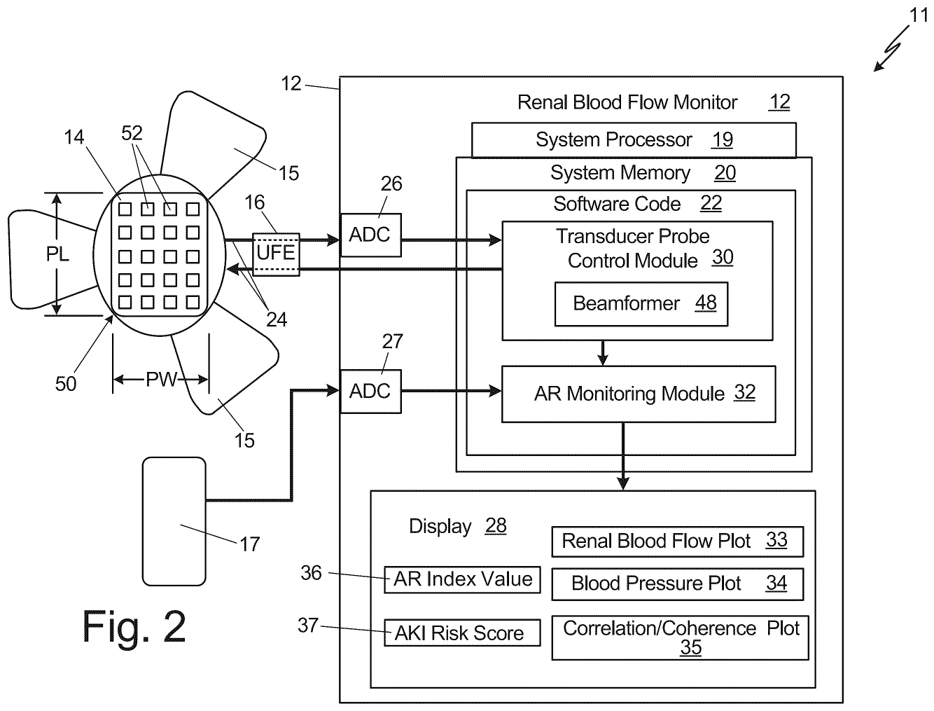

As shown in FIG. 1, system memory 20 of blood flow monitor 12 can store software code 22 which forms a monitoring model of blood flow monitor 12. Software code 22 can include transducer probe control module 30 for controlling and commanding ultrasound transducer probe 14. Transducer probe control module 30, as discussed in greater detail below with reference to FIG. 2, includes a beamformer that keeps ultrasound transducer probe 14 aimed at the renal blood flow of kidney 42L so that ultrasound transducer probe 14 continuously senses and communicates the Doppler flow signal of the renal blood flow to blood flow monitor 12 throughout the surgery, medical procedure, or medical observation of patient 10. Software code 22 can also include AR monitoring module 32 which includes monitoring software code to continuously monitor the doppler flow signal DF of the renal blood flow and continuously monitor the arterial pressure of patient 10 during the surgery, medical procedure, or medical observation of patient 10 to determine an autoregulation profile of the renal blood flow of kidney 42L. The autoregulation profile of the renal blood flow of kidney 42L, as will be discussed in greater detail below, is based on a calculated mathematical relationship between the renal blood flow of kidney 42L and the arterial pressure of patient 10. AR monitoring module 32 can also include code to determine an acute kidney injury (AKI) risk score of patient 10 from the autoregulation profile of the renal blood flow of kidney 42L. The AKI risk score represents the probability that kidney 42L is experiencing or approaching an acute kidney injury. When monitoring system 11 is used to monitor an organ other than kidneys 42L and 42R of patient 10, AR monitoring module 32 can be adapted to determine an autoregulation profile and a real-time organ injury risk score from the arterial pressure of patient 10 and a Doppler flow signal of the organ blood flow of the organ that is being monitored, such as liver 44.

System processor 19 is a hardware processor configured to execute software code 22, which implements transducer probe control module 30 and AR monitoring module 32. Examples of system processor 19 can include any one or more of a microprocessor, a controller, a digital signal processor (DSP), an application specific integrated circuit (ASIC), a field-programmable gate array (FPGA), or other equivalent discrete or integrated logic circuitry.

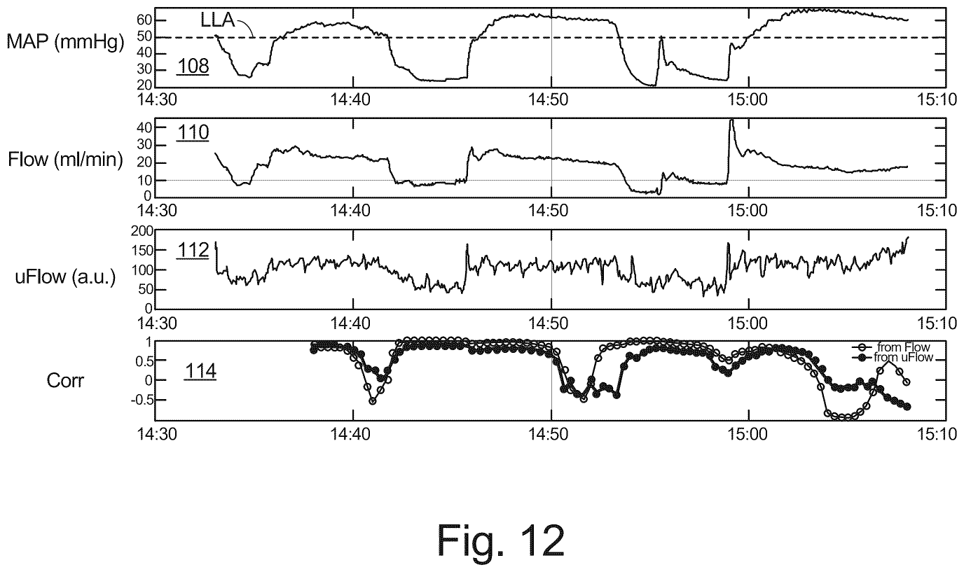

Display 28 provides user interface 29, which includes control elements that enable user interaction with blood flow monitor 12 and/or other components of monitoring system 11. Display 28 is in communication with system processor 19 and is configured to provide first plot 33, second plot 34, and third plot 35. First plot 33 can be a plot of the Doppler flow signal of the renal blood flow of kidney 42L over time, a plot over time of the flow rate of the renal blood flow determined from the Doppler flow signal of the renal blood flow, or a plot of the change in the flow rate of the renal blood flow of kidney 42L over time. Second plot 34 can be a plot of the arterial pressure of patient 10 over time, or a plot of the change in arterial pressure of patient 10 over time. Third plot 35 can be a plot of the calculated mathematical relationship between the renal blood flow of kidney 42L and the arterial pressure of patient 10 over time that forms the autoregulation profile of the renal blood flow of kidney 42L, such as shown in plot 114 of FIG. 12. In other examples, third plot 35 can also include a plot of the calculated mathematical relationship versus the arterial pressure of patient 10 with each data point color coded to represent time. In addition to showing plots 33, 34, and 35, display 28 can also provide an audible representation of any of plots 33, 34, and 35 via a speaker or simply display the numerical values of plots 33, 34, and 35, such as through a table.

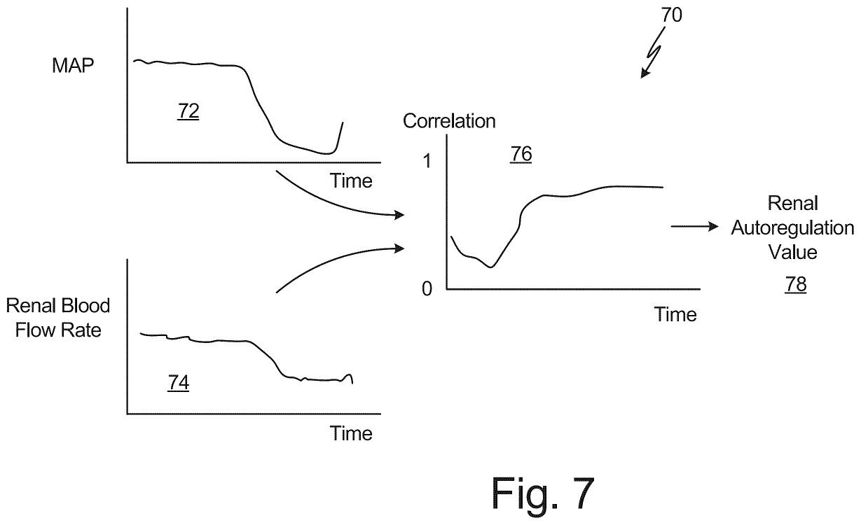

Display 28, as shown in FIG. 1, also shows autoregulation index value 36 and injury score indicator 37. Autoregulation index value 36 is a representation of the real-time value or state of the autoregulation profile of patient 10 based upon a calculated mathematical relationship between the renal blood flow of kidney 42L and the arterial pressure of patient 10. As discussed in greater detail below with reference to FIGS. 7-10, the calculated mathematical relationship between the renal blood flow of kidney 42L and the arterial pressure of patient 10 can be a correlation or a coherence between a flow rate of the renal blood flow of kidney 42L and the arterial pressure of patient 10. Autoregulation index value 36 is an inverse to the correlation or the coherence between the flow rate of the renal blood flow of kidney 42L and the arterial pressure of patient 10. Injury score indicator 37 is a representation of the real-time AKI risk score of patient 10 determined from the autoregulation index values by system processor 19 and AR monitoring module 32. Display 28 can also include a sensory alarm to alert medical personnel when autoregulation index value 36 of the renal blood flow of kidney 42L approaches a lower limit of autoregulation or an upper limit of autoregulation. As discussed in greater detail below, the lower limit of autoregulation is a mean arterial pressure (MAP) value below which the autoregulation of the renal blood flow of kidney 42L becomes impaired. The upper limit of autoregulation is a MAP value above which the autoregulation of the renal blood flow of kidney 42L becomes impaired. The sensory alarm can also alert medical personnel when the real-time AKI risk score of patient 10 is approaching or exceeding a predetermined threshold. The sensory alarm can be implemented as one or more of a visual alarm, an audible alarm, a haptic alarm, or other type of sensory alarm. For instance, the sensory alarm can be invoked as any combination of flashing and/or colored graphics shown by user interface 29 on display 28, a warning sound such as a siren or repeated tone, and a haptic alarm configured to cause blood flow monitor 12 to vibrate or otherwise deliver a physical impulse perceptible to medical personnel.

Display 28 can be a liquid crystal display (LCD), a light-emitting diode (LED) display, an organic light-emitting diode (OLED) display, or other display device suitable for providing information to users in graphical form. User interface 29 can include graphical and/or physical control elements that enable user input to interact with blood flow monitor 12 and/or other components of monitoring system 11. In some examples, user interface 29 can take the form of a graphical user interface (GUI) that presents graphical control elements presented at, e.g., a touch-sensitive and/or pressure sensitive display screen of display 28. In such examples, user input can be received in the form of gesture input, such as touch gestures, scroll gestures, zoom gestures, or other gesture input. In certain examples, user interface 29 can take the form of and/or include physical control elements, such as a physical buttons, keys, knobs, or other physical control elements configured to receive user input to interact with components of monitoring system 11. User interface 29 can include a speaker that allows blood flow monitor 12 the ability to generate an audible alarm.

In operation of monitoring system 11, before a surgery, medical procedure, or medical observation begins, a medical worker connects hemodynamic pressure sensor 17 to patient 10. In the example of FIG. 1, the medical worker connects hemodynamic pressure sensor 17 to patient 10 by first inserting radial arterial catheter 18 into the arm of patient 10 and then connecting hemodynamic pressure sensor 17 to radial arterial catheter 18. In other examples, the medical worker can connect the hemodynamic pressure sensor 17 to patient 10 by first inserting a femoral arterial catheter into the leg of patient 10 and then connecting the hemodynamic pressure sensor 17 to the femoral arterial catheter. In other examples, the medical worker can connect the hemodynamic pressure sensor 17 non-invasively on an extremity of patient 10, such as a wrist, an arm, a finger, an ankle, a toe, or other extremity of patient 10. Once hemodynamic pressure sensor 17 is connected to patient 10, hemodynamic pressure sensor senses hemodynamic data representative of the arterial pressure of patient 10 and communicates the hemodynamic data (e.g., as analog sensor data), to blood flow monitor 12. Second ADC 27 converts the analog hemodynamic data to digital hemodynamic data representative of the arterial pressure of patient 10. System processor 19 of blood flow monitor 12 receives the hemodynamic data representative of the arterial pressure of patient 10 and processes the hemodynamic data representative of the arterial pressure through AR monitoring module 32.

Before the surgery, the medical procedure, or the medical observation begins, the medical worker also places ultrasound transducer probe 14 on abdomen 40 of patient 10. The medical worker uses ultrasound transducer probe 14 to locate the Doppler flow signal of the renal blood flow of kidney 42L. Ultrasound transducer probe 14 can generate an audible representation of the Doppler flow signal to assist the medical worker in locating the Doppler flow signal of the renal blood flow of kidney 42L. Once the medical worker finds the Doppler flow signal of the renal blood flow of kidney 42L, the medical worker attaches and secures ultrasound transducer probe 14 to patient 10 with adhesive patch 15. Adhesive patch 15 keeps ultrasound transducer probe 14 in constant contact with patient 10 such that ultrasound transducer probe 14 does not shift positions on patient 10 during the surgery, medical procedure, or medical observation and lose the Doppler flow signal of the renal blood flow of kidney 42L. Ultrasound transducer probe 14 relays the received ultrasound signals to blood flow monitor 12 via cable(s) 24 or wirelessly. In the case of wireless transmission, the ultrasound transducer probe 14 includes the UFE circuitry 16. System processor 19 of blood flow monitor 12 receives the Doppler flow signal and processes the Doppler flow signal sequentially or simultaneously through transducer probe control module 30 and AR monitoring module 32.

System processor 19 can execute the monitoring software code of AR monitoring module 32 to continuously monitor the Doppler flow signal of the renal blood flow sensed by ultrasound transducer probe 14 and to continuously monitor the arterial pressure of patient 10 sensed by hemodynamic pressure sensor 17 throughout a duration of the surgery, medical procedure, or medical observation of patient 10. System processor 19 also executes the monitoring software code of AR monitoring module 32 to calculate a mathematical relationship between the renal blood flow of kidney 42L and the arterial pressure of patient 10 and to use that mathematical relationship to generate the autoregulation profile of the renal blood flow of kidney 42. System processor 19 also executes AR monitoring module 32 to continuously monitor the autoregulation profile of the renal blood flow of kidney 42L and to estimate the AKI risk score of kidney 42L of patient 10 from the autoregulation values.

System processor 19 outputs information to display 28 to generate first plot 33, second plot 34, and third plot 35. First plot 33 can be a plot of the Doppler flow signal of the renal blood flow of kidney 42L over time, a plot over time of the flow rate of the renal blood flow determined by system processor 19 from the Doppler flow signal of the renal blood flow, or a plot of the change in the flow rate of the renal blood flow of kidney 42L over time. Second plot 34 can be a plot of the arterial pressure of patient 10 over time, or a plot of the change in arterial pressure of patient 10 over time. Third plot 35 can be a plot of the calculated mathematical relationship between the renal blood flow of kidney 42L and the arterial pressure of patient 10 over time or a plot of the autoregulation profile of the renal blood flow of kidney 42L that system processor 19 determined from the renal blood flow of kidney 42L and the arterial pressure of patient 10. System processor 19 also outputs autoregulation index value 36 and injury score indicator 37. As previously discussed, autoregulation index value 36 is a representation of the real-time value or state of the autoregulation profile of the renal blood flow of kidney 42L, and injury score indicator 37 is a representation of the real-time AKI risk score of patient 10.

As the surgery, medical procedure, or medical observation of patient 10 progresses, system processor 19 continues to receive the Doppler flow signal from ultrasound transducer probe 14, continues to receive the hemodynamic data representative of the arterial pressure of patient 10, continues to calculate the mathematical relationship between the renal blood flow of kidney 42L and the arterial pressure of patient 10, continues to output plots 33, 34, and 35 to display 28, continues to output autoregulation index value 36 to display 28, and continues to output injury score indicator 37 to display 28. If autoregulation index value 36 changes toward an undesired threshold, such as trending toward the lower limit of autoregulation or the upper limit of autoregulation, system processor 19 and display 28 can alert the medical personnel so that the medical personnel can act to restore normal autoregulation of the renal blood flow of kidney 42L. For example, medical personnel can administer medication or fluids that increases the arterial pressure of patient 10 to raise and/or maintain autoregulation index value 36 above the lower limit of autoregulation. In another example, medical personnel can administer medication or take action to reduce the arterial pressure of patient 10 to lower and/or maintain autoregulation index value 36 below an upper limit of autoregulation.

Similarly, if the real-time AKI risk score of kidney 42L changes toward an undesired threshold, or changes at an undesired rate, system processor 19 and display 28 can alert the medical personnel so that the medical personnel can take action to increase kidney perfusion and prevent AKI to kidney 42L, or minimize AKI to kidney 42L. For example, medical personnel can administer medication or fluids that increases the renal blood flow and perfusion to kidney 42L or improves autoregulation of the renal blood flow to kidney 42L. At the end of the surgery, the medical procedure, or the medical observation, system processor 19 and AR monitoring module 32 can estimate a final AKI risk score for kidney 42L and output the final AKI risk score to display 28. If the final AKI risk score for kidney 42L indicates that kidney 42L has a high risk of AKI, medical personnel can take immediate action to treat kidney 42L without having to wait for biomarkers to appear in blood and urine samples of patient 10. Biomarkers that indicate AKI can take several hours or days to appear in blood and urine samples of patient 10. With monitoring system 11, the medical personnel can determine quickly whether patient 10 needs to be treated for AKI of kidney 42L.

If kidney 42L of patient 10 moves within abdomen 40 of patient 10 during the surgery, medical procedure, or medical observation, transducer probe control module 30 will detect a change in the Doppler flow signal and will respond by adjusting the focusing location of the set of beams to scan abdomen 40 of patient 10 to relocate the Doppler flow signal of the renal blood flow of kidney 42L. As discussed below with reference to FIGS. 2-5, blood flow monitor 12 can include a beamformer that can steer beam signals produced by an array of transducer elements of ultrasound transducer probe 14.

FIG. 2 is another schematic diagram of blood flow monitor 12. As shown in FIG. 2, blood flow monitor 12 can include beamformer 48 and ultrasound transducer probe 14 can include array 50 of transducer elements 52. Each transducer element 52 of array 50 can comprise a piezoelectric material, such as lead zirconate titanate, capable of transmitting ultrasound pulses and detecting ultrasound pulses. Array 50 of transducer elements 52 of ultrasound transducer probe 14 can form a two-dimensional phased array with probe length PL and probe width PW. As a phased array, each transducer element 52 in array 50 can pulse individually relative the other transducer elements 52 in array 50.

In the example of FIG. 2, beamformer 48 drives array 50 of transducer elements 52 via system processor 19 and UFE circuitry 16. Beamformer 48 functions as a transducer probe controller with flow signal tracking software code that controls the timing that each transducer element 52 in array 50 emits an ultrasound pulse. Beamformer 48 can time and pattern when each transducer element 52 emits a pulse such that array 50 can form one or more ultrasonic beams and can sweep or steer the one or more ultrasonic beams without physically moving the position of ultrasound transducer probe 14 on patient 10. Beamformer 48 can be a software sub-module of transducer probe control module 30 that can be executed by system processor 19 to control activation of transducer elements 52 of array 50. In other examples, beamformer 48 can be a separate hardware component from system processor 19 and system memory 20 with separate memory and software from software code 22 that coordinates with system processor 19 to control activation of transducer elements 52 of array 50. In the example of FIG. 2, beamformer 48 is housed within blood flow monitor 12 as part of transducer probe control module 30 of software code 22 that is executed by system processor 19. In other examples, beamformer 48 can be fully or partially housed within a casing of ultrasound transducer probe 14 as a separate hardware and software unit that coordinates with system processor 19. Housing beamformer 48 in the same unit as blood flow monitor 12 (whether as part of software code 22 or as an add-on hardware component) can decrease the overall size and thickness of ultrasound transducer probe 14. Ultrasound transducer probe 14 can be relatively thin and flat in profile, with a thickness that is smaller than a width or diameter of ultrasound transducer probe 14. Attaching ultrasound transducer probe 14 to patient 10 by adhesive patch 30 is easier and more secure when ultrasound transducer probe 14 has a thin and flat profile.

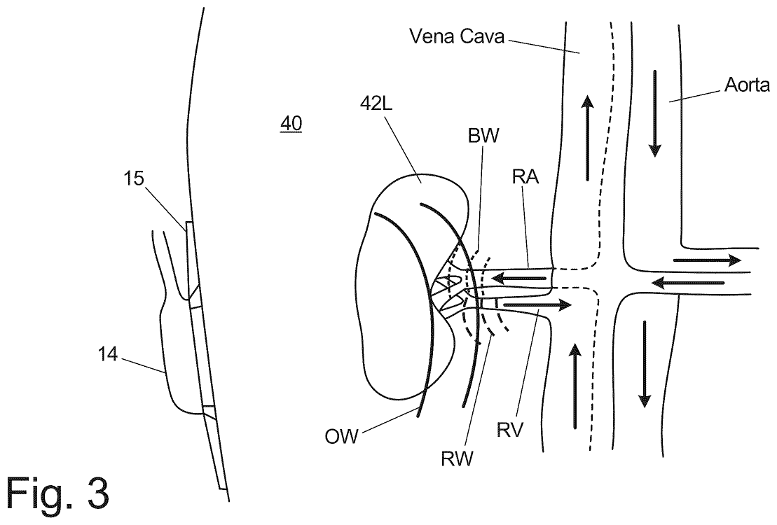

FIG. 3 is another schematic diagram of ultrasound transducer probe 14 attached to abdomen 40 of patient 10 by adhesive patch 15 over kidney 42L. The Doppler flow signal of kidney 42L can be measured from either the renal artery RA as blood enters kidney 42L from the aorta of patient 10 via the renal artery, or from the renal vein RV as blood exits kidney 42L to the vena cava of patient 10 via the renal vein RV. Ultrasound transducer probe 14 generates originating signals OW that move into abdomen 40 of patient 10. Due to Doppler physics, a Doppler signal BW of the blood flow in the renal artery RA is “blue shifted” and thus appears to have a shorter wavelength than a send signal of the ultrasound transducer probe 14 as the blood flow in the renal artery RA is moving toward the ultrasound transducer probe 14. A Doppler signal RW of the blood flow in the renal vein RV is “red shifted” and thus appears to have a longer wavelength than the send signal of the ultrasound transducer probe 14 as the blood flow in the renal vein RV is moving away from the ultrasound transducer. Since the Doppler signal BW is blue shifted and the Doppler signal RW is red shifted, blood flow monitor 12 can easily distinguish renal artery blood flow from renal vein blood flow. In human subjects the renal artery RA and renal vein RV are close and aligned parallel to one another such that beamformer 48 can position the beam(s) to capture both arterial and venous flow of kidney 42L simultaneously.

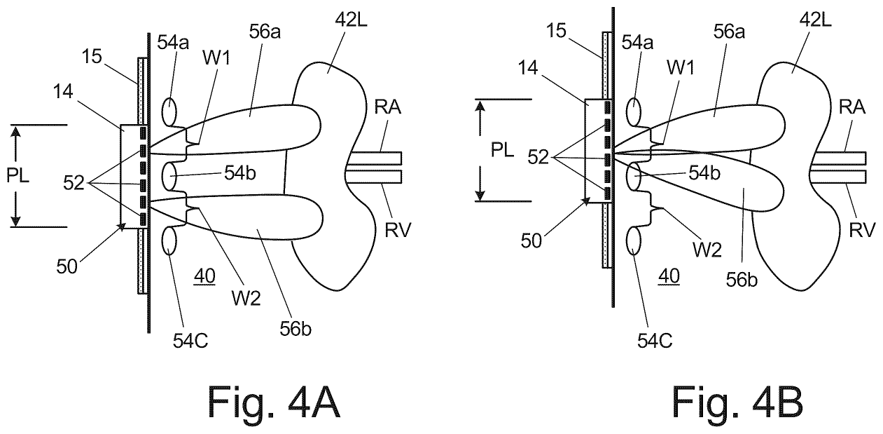

FIGS. 4A and 4B will be discussed concurrently. FIG. 4A is another schematic diagram of ultrasound transducer probe 14 attached to abdomen 40 of patient 10 by adhesive patch 15 over kidney 42L. FIG. 4B is also a schematic diagram of ultrasound transducer probe 14 attached to abdomen 40 of patient 10 by adhesive patch 15 over kidney 42L. In the example of FIGS. 4A and 4B, ultrasound transducer probe 14 is attached by adhesive patch 15 to a surface of abdomen 40 over kidney 42L and over at least some of ribs 54a, 54b, and 54c of patient 10.

Ultrasound transducer probe 14 can include a probe length PL, probe width PW (shown in FIG. 2), or diameter that is large enough that array 50 of transducer elements 52 of ultrasound transducer probe 14 can cover one or more acoustic windows in patient 10. An acoustic window of patient 10 is defined as an area of patient 10 where transmission of ultrasonic waves is not substantially attenuated in comparison to immediate surroundings. For example, array 50 of transducer elements 52 of ultrasound transducer probe 14 can be sized in length or width to extend over at least two intercostal spaces of patient 10. For example, in FIG. 4A, array 50 of transducer elements 52 of ultrasound transducer probe 14 is positioned over first acoustic window W1 (formed by the intercostal space between rib 54a and rib 54b) and over second acoustic window W2 (formed by the intercostal space between rib 54b and rib 54c). In the example of FIG. 4A, beamformer 48 (shown in FIG. 2) can selectively activate transducer elements 52 in array 50 to steer signal beams 56a and 56b into abdomen 40 through the first acoustic window W1 and/or second acoustic window W2 to avoid ribs 54a, 54b, and 54c. In the example of FIG. 4B, ultrasound transducer probe 14 is positioned slightly higher on abdomen 40 of patient 10 in comparison to the example of FIG. 4A. However, the probe length PL or probe width PW of ultrasound transducer probe 14 is long enough that ultrasound transducer probe 14 still has access to first acoustic window W1 and can still scan and steer signal beams 56a and 56b into abdomen 40 through the first acoustic window W1. Regardless of where ultrasound transducer probe 14 is placed over ribs 54a, 54b, and 54c, ribs 54a, 54b, and 54c will not block the direct view of kidney 42L from array 50 of ultrasound transducer probe 14.

Beamformer 48 controls transducer elements 52 in array 50 to electronically beam scan abdomen 40 to find and sense the Doppler flow signal when ultrasound transducer probe 14 is first placed on patient 10. Beamformer 48 also controls transducer elements 52 in array 50 to track scan abdomen 40 to track the Doppler flow signal of the renal blood flow over time. Beamformer 48 beam scans and/or track scans the Doppler flow signal of the renal blood flow of kidney 42L of patient 10 by sequentially emitting signal beams 56a and 56b from array 50 of transducer elements 52 and focusing each of beams 56a and 56b in different locations. Signal beams 56a and 56b track the Doppler flow signal relative to array 50 of transducer elements 52. If kidney 42L, renal artery RA, and/or renal vein RV shifts within abdomen 40, the Doppler flow signal of the renal blood flow can be altered and decrease in signal strength. If that should happen, beamformer 48 can emit signal beam 56a and signal beam 56b (and possibly more signal beams) to scan and sweep about abdomen 40. In one example, beamformer 48 uses signal beams 56a and 56b to track a center of the renal blood flow where the Doppler flow signal is strongest and adjusts signal beams 56a and 56b to follow the center of the renal blood flow when the center moves and changes position. While beamformer 48 is track scanning the Doppler flow signal to increase signal strength, system processor 19 can cease to calculate the mathematical relationship between the renal blood flow of kidney 42L and the arterial pressure of patient 10 until the signal strength of the Doppler flow signal increases.

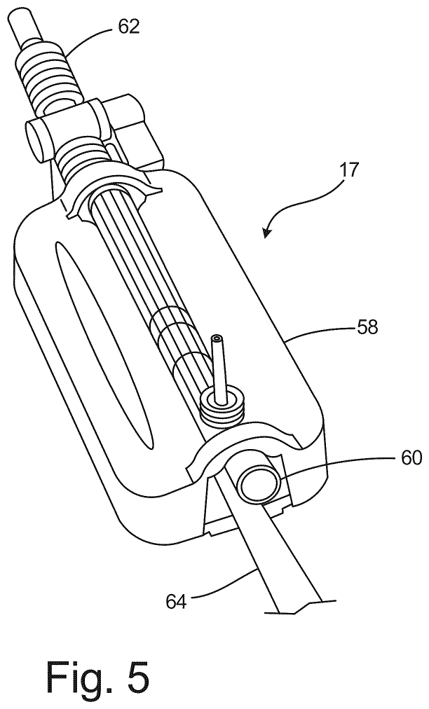

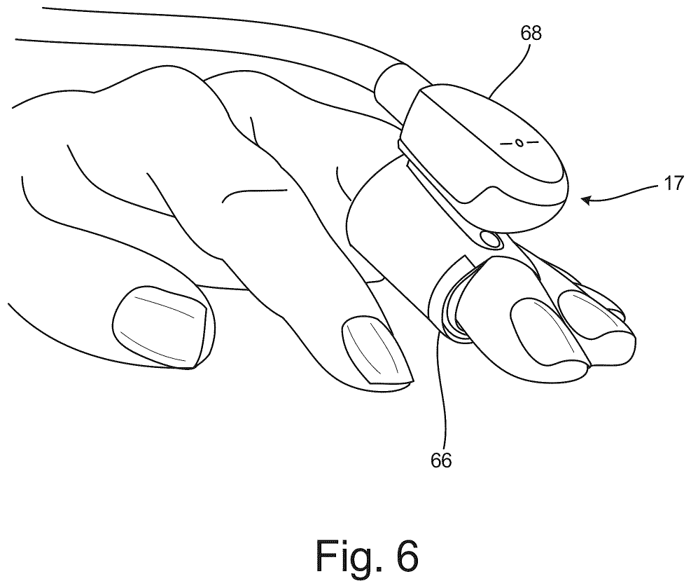

In order for ultrasound transducer probe 14 to measure the Doppler flow signal of the renal blood flow of kidney 42L, ultrasound transducer probe 14 can have a low center frequency between 0.5 MHz and 4.0 MHz. With a center frequency between 0.5 MHz and 4.0 MHZ, ultrasound transducer probe 14 can penetrate more than 15 cm into patient 10, which is a sufficient depth to measure the renal blood flow. This depth also allows ultrasound transducer probe 14 the ability to measure hepatic blood flow, celiac blood flow, portal blood flow, and mesenteric blood flow. Monitoring system 11 does not use ultrasound transducer probe 14 for high resolution imaging of kidney 42L. Thus, ultrasound transducer probe 14 can have a lower transducer element count than an ultrasound transducer probe used for ultrasound imaging. Lowering the transducer element count of array 50 of transducer elements 52 increases a signal-to-noise ratio (SNR) of the Doppler flow signal of the renal blood flow sensed by ultrasound transducer probe 14. Various embodiments of hemodynamic pressure sensor 17 are discussed in greater detail with reference to FIGS. 5 and 6.

FIG. 5 is a perspective view of hemodynamic pressure sensor 17 that can be attached to the patient for sensing hemodynamic data representative of the arterial pressure of patient 10. Hemodynamic pressure sensor 17, illustrated in FIG. 5, is one example of a minimally invasive hemodynamic pressure sensor that can be attached to patient 10 via radial arterial catheter 18 inserted into an arm of patient 10, as shown in FIG. 1. In other examples, hemodynamic pressure sensor 17 can be attached to patient 10 via a femoral arterial catheter inserted into a leg of patient 10.

As illustrated in FIG. 5, hemodynamic pressure sensor 17 includes housing 58, fluid input port 60, catheter-side fluid port 62, and Input/Output (I/O) cable 64. Fluid input port 60 is configured to be connected via tubing or other hydraulic connection to a fluid source, such as a saline bag or other fluid input source. Catheter-side fluid port 62 is configured to be connected via tubing or other hydraulic connection to a catheter (e.g., radial arterial catheter 18 or a femoral arterial catheter) that is inserted into an arm of patient 10 (i.e., radial arterial catheter 18) or a leg of patient 10 (i.e., a femoral arterial catheter). I/O cable 64 connects hemodynamic pressure sensor 17 to blood flow monitor 12 via, e.g., one or more of I/O connectors. Housing 58 of hemodynamic pressure sensor 17 encloses one or more pressure transducers, communication circuitry, processing circuitry, and corresponding electronic components to sense fluid pressure corresponding to arterial pressure of patient 10 that is transmitted to blood flow monitor 12 via I/O cable 64.

In operation, a column of fluid (e.g., saline solution) is introduced from a fluid source (e.g., a saline bag) through hemodynamic pressure sensor 17 via fluid input port 60 to catheter-side fluid port 62 toward the catheter inserted into patient 10. Arterial pressure is communicated through the fluid column to pressure sensors located within housing 58 which sense the pressure of the fluid column. Hemodynamic pressure sensor 17 translates the sensed pressure of the fluid column to an electrical signal via the pressure transducers and outputs the corresponding electrical signal to blood flow monitor 12 via I/O cable 64. Hemodynamic pressure sensor 17 therefore transmits analog sensor data (or a digital representation of the analog sensor data) to blood flow monitor 12 that is representative of substantially continuous beat-to-beat monitoring of the arterial pressure of patient 10.

FIG. 6 is a perspective view of an alternative example of hemodynamic pressure sensor 17 for sensing hemodynamic data representative of arterial pressure of patient 10. Hemodynamic pressure sensor 17, illustrated in FIG. 6, is one example of a non-invasive hemodynamic pressure sensor that can be attached to patient 10 via one or more finger cuffs to sense data representative of arterial pressure of patient 10. As illustrated in FIG. 6, hemodynamic pressure sensor 17 includes inflatable finger cuff 66 and heart reference sensor 68. Inflatable finger cuff 66 includes an inflatable blood pressure bladder configured to inflate and deflate as controlled by a pressure controller (not illustrated) that is pneumatically connected to inflatable finger cuff 66. Inflatable finger cuff 66 also includes an optical (e.g., infrared) transmitter and an optical receiver that are electrically connected to the pressure controller (not illustrated) to measure the changing volume of the arteries under the cuff in the finger.

In operation, the pressure controller continually adjusts pressure within the finger cuff to maintain a constant volume of the arteries in the finger (i.e., the unloaded volume of the arteries) as measured via the optical transmitter and optical receiver of inflatable finger cuff 66. The pressure applied by the pressure controller to continuously maintain the unloaded volume is representative of the blood pressure in the finger and is communicated by the pressure controller to blood flow monitor 12 shown in FIG. 1. Heart reference sensor 68 measures the hydrostatic height difference between the level at which the finger is kept and the reference level for the pressure measurement, which typically is heart level. Accordingly, hemodynamic pressure sensor 17 transmits hemodynamic data that is representative of substantially continuous beat-to-beat monitoring of the arterial pressure of patient 10. As discussed below with reference to FIGS. 7-10, hemodynamic pressure sensor 17 transmits the hemodynamic data to system processor 19 where system processor 19 calculates a mathematical relationship between the arterial pressure of patient 10 and the renal blood flow of kidney 42L of patient 10.

FIG. 7 is a diagrammatic representation of method 70 for determining in a time domain the mathematical relationship between the arterial pressure of patient 10 and the renal blood flow of kidney 42L of patient 10. Method 70 in FIG. 7 is described by first data plot 72, second data plot 74, and correlation plot 76. First data plot 72 represents changes in a mean arterial pressure (MAP) of patient 10 over time that are determined by system processor 19 from the hemodynamic data sensed by hemodynamic pressure sensor 17 in real time. System processor 19 can output first data plot 72 to display 28 (shown in FIGS. 1 and 2) as second plot 34. Second data plot 74 represents changes in a flow rate of the renal blood flow of kidney 42L over time that are estimated by system processor 19 from the Doppler flow signal of the renal blood flow sensed by ultrasound transducer probe 14 in real time. System processor 19 can estimate the flow rate of the renal blood flow from the Doppler flow signal of the renal blood flow by using the flow velocity of the Doppler flow signal and an average cross-sectional area of the renal artery RA and/or the renal vein RV of patient 10. System processor 19 can output second data plot 74 to display 28 as first plot 33 (shown in FIGS. 1 and 2). In other examples, the flow rate of the renal blood flow can be determined from the Doppler flow signal by using a flow velocity signal, using a peak flow velocity signal, and/or using a renal blood flow relative change signal.

Correlation plot 76 represents the calculated mathematical relationship over time that system processor 19 determines and evaluates between changes in the MAP and changes in the flow rate of the renal blood flow of kidney 42L. The calculated mathematical relationship shown in FIG. 7 is a correlation or non-correlation between the changes in the MAP and changes in the flow rate of the renal blood flow of kidney 42L. System processor 19 can use a Pearson correlation coefficient computed over a rolling window of time to determine the correlation or the non-correlation between the changes in the arterial pressure and the changes in the flow rate of the renal blood flow of kidney 42L. System processor 19 and AR monitoring module 32 (shown in FIGS. 1 and 2) can use Equation 1 below to determine the Pearson correlation coefficient between the changes in the MAP and changes in the flow rate of the renal blood flow of kidney 42L:

$$r = \frac{\sum ( {x}_{i} - \overline{x} ) ( {y}_{i} - \overline{y} )}{\sqrt{\sum {( {x}_{i} - \overline{x} )}^{2} \sum {( {y}_{i} - \overline{y} )}^{2}}} . \qquad Equation 1$$

where r is the correlation coefficient between the changes in the MAP and changes in the flow rate of the renal blood flow of kidney 42L, xᵢ is the real-time value of the MAP, and x is the running mean of the values of the MAP over the rolling time window. The variable yᵢ is the real time value of the flow rate of the renal blood flow of kidney 42L estimated by system processor 19, and y is the running mean of the values of the flow rate of the renal blood flow of kidney 42L over the rolling time window.

As system processor 19 and AR monitoring module 32 determine the correlation coefficient between the changes in the MAP and the changes in the flow rate of the renal blood flow of kidney 42L, system processor 19 can generate correlation plot 76 and can output correlation plot 76 to display 28 as third plot 35 (shown in FIGS. 1 and 2). System processor 19 and AR monitoring module 32 use the correlation coefficient between the changes in the MAP and the changes in the flow rate of the renal blood flow of kidney 42L to generate renal autoregulation value 78. Renal autoregulation value 78 is a real-time value or state of the autoregulation of patient 10. When the correlation coefficient between the changes in the MAP and the changes in the flow rate of the renal blood flow of kidney 42L is high (e.g., approaching a value of 1), renal autoregulation value 78 is low or indicates that the autoregulation of the renal blood flow of kidney 42L is impaired. When the correlation coefficient between the changes in the MAP and the changes in the flow rate of the renal blood flow of kidney 42L is low (e.g., below 0.5), renal autoregulation value 78 is high or indicates that the autoregulation of the renal blood flow of kidney 42L is normal. System processor 19 can output renal autoregulation value 78 to display 28 as autoregulation index value 36 shown in FIGS. 1 and 2.

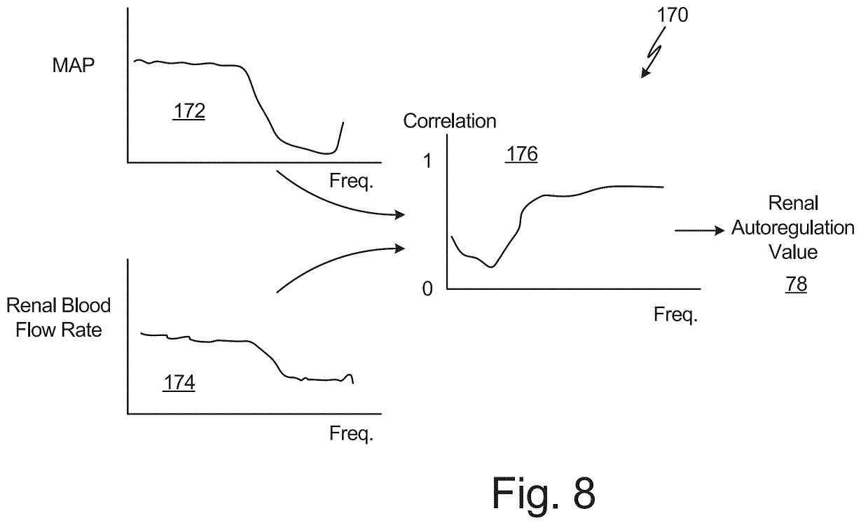

In other examples, system processor 19 and AR monitoring module 32 can use mathematical correlations or tools other than the Pearson correlation coefficient to determine the calculated mathematical relationship between the changes in the arterial pressure and the changes in the flow rate of the renal blood flow of kidney 42L over the course of the surgery, the medical procedure, or the medical observation of the patient. For example, as shown in FIG. 8, system processor 19 and AR monitoring module 32 can use a Coherence function computed across a prespecified frequency range and computed from parameters of a transfer function of the MAP signal and a transfer function of the flow rate of the renal blood flow. A Fourier transformation can be used as the transfer function to transform the MAP signal of patient 10 (shown in first data plot 72 of FIG. 7) from the time domain to a frequency domain, as represented by first data plot 172 in FIG. 8. System processor 19 can output first data plot 172 to display 28 (shown in FIGS. 1 and 2) as second plot 34. Similarly, a Fourier transformation can be used as the transfer function to transform the flow rate of the renal blood flow (shown in second data plot 74 of FIG. 7) from the time domain to a frequency domain, as represented by second data plot 174 in FIG. 8. System processor 19 can output second data plot 174 to display 28 as first plot 33 (shown in FIGS. 1 and 2).

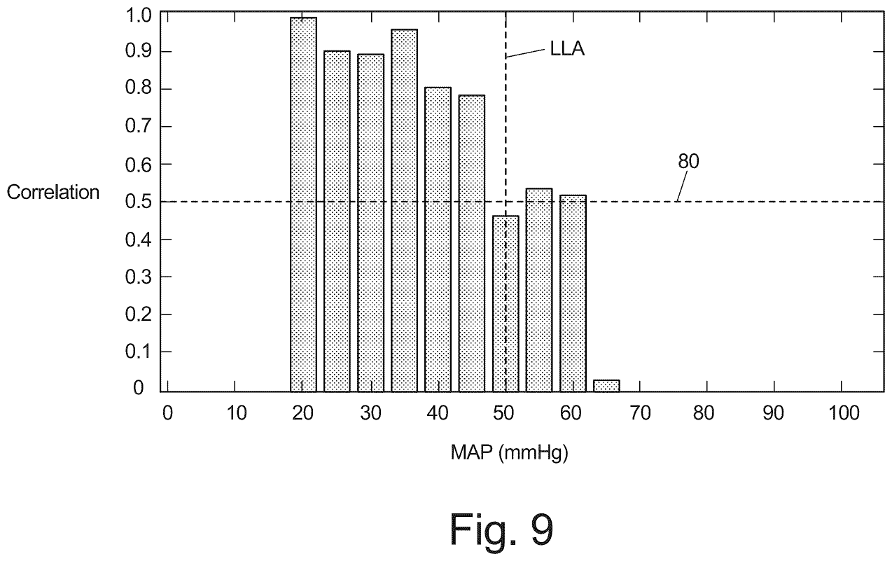

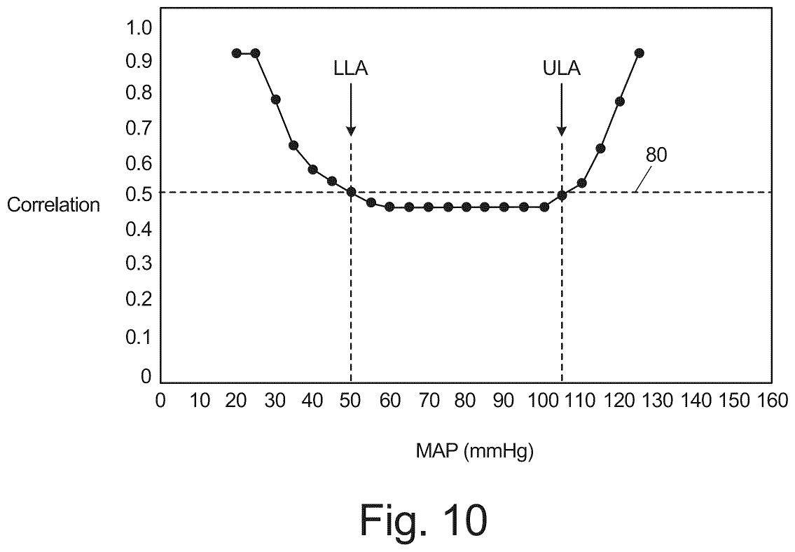

System processor 19 and AR monitoring module 32 input the transformed flow rate of the renal blood flow of kidney 42L and the transformed MAP of patient 10 into the Coherence function to generate a coherence coefficient between the transformed flow rate of the renal blood flow of kidney 42L and the transformed MAP of patient 10, as represented by coherence plot 176 in FIG. 8. System processor 19 can output coherence plot 176 to display 28 as third plot 35 (shown in FIGS. 1 and 2). Similar to the correlation coefficient described with reference to FIG. 7, system processor 19 and AR monitoring module 32 can use the coherence coefficient to generate renal autoregulation value 78. Renal autoregulation value 78 is a real-time value or state of the autoregulation of patient 10. When the coherence coefficient between the changes in the MAP and the changes in the flow rate of the renal blood flow of kidney 42L is high (e.g., approaching a value of 1), renal autoregulation value 78 is low or indicates that the autoregulation of the renal blood flow of kidney 42L is impaired. When the correlation coefficient between the changes in the MAP and the changes in the flow rate of the renal blood flow of kidney 42L is low (e.g., below a predetermined coherence threshold), renal autoregulation value 78 is high or indicates that the autoregulation of the renal blood flow of kidney 42L is normal. System processor 19 can output renal autoregulation value 78 to display 28 as autoregulation index value 36 shown in FIGS. 1 and 2. As discussed below with reference to FIGS. 9 and 10, system processor 19 and AR monitoring module 32 use the correlation coefficient and/or the coherence coefficient between the changes in the MAP and the changes in the flow rate of the renal blood flow of kidney 42L to determine and monitor the autoregulation profile of patient 10.

FIG. 9 is a chart with the X-axis divided into MAP bins of 5 mmHg increments and the correlation coefficient from FIG. 7 set as the Y-axis. As system processor 19 determines and monitors the correlation coefficient between the flow rate of the renal blood flow of kidney 42L and the MAP of patient 10 over time, system processor 19 can generate the chart of FIG. 9 by sorting the values of the correlation coefficient into the MAP bins of the chart of FIG. 9 to generate an autoregulation profile or index of patient 10. System processor 19 will only sort values of the correlation coefficient into the MAP bins of the chart of FIG. 9 when ultrasound transducer probe 14 and beamformer 48 have a steady and stable reading of the Doppler flow signal of the renal blood flow of kidney 42L. When beamformer 48 and ultrasound transducer probe 14 are searching for the Doppler flow signal, or when the Doppler flow signal is unreliable, or if patient movement causes an artifact that disrupts the Doppler flow signal, system processor 19 does not add values of the correlation coefficient to the MAP bins of the chart of FIG. 9. Similarly, if hemodynamic pressure sensor 17 generates unreliable readings of the arterial pressure of patient 10, system processor 19 does not add values of the correlation coefficient to the MAP bins of the chart of FIG. 9 until hemodynamic pressure sensor 17 can regain reliable readings of the arterial pressure of patient 10.

The chart of FIG. 9 includes correlation threshold line 80 at about the correlation coefficient value of 0.5. When the correlation coefficient of any given MAP value is above correlation threshold line 80, the autoregulation of the renal blood flow of kidney 42L can be described as being passive, and the passiveness of the autoregulation of the renal blood flow of kidney 42L increases as the correlation coefficient approaches a value of 1. When the autoregulation of the renal blood flow of kidney 42L is passive, the autoregulation of the renal blood flow of kidney 42L is impaired and the flow rate of the renal blood flow of kidney 42L fluctuates with the MAP of patient 10. Thus, when the correlation coefficient indicates that changes in the flow rate of the renal blood flow of kidney 42L correlate with the MAP of patient 10, autoregulation of the renal blood flow of kidney 42L is impaired.

When the correlation coefficient of any given MAP value is below correlation threshold line 80, the autoregulation of the renal blood flow of kidney 42L has substantially normal function. When the autoregulation of the renal blood flow of kidney 42L is functioning normally, the flow rate of the renal blood flow of kidney 42L is independent of the MAP of patient 10. Thus, changes in the flow rate of the renal blood flow of kidney 42L do not correlate with changes in the MAP of patient 10 when autoregulation of the renal blood flow of kidney 42L is functioning normally. The degree to which the autoregulation of the renal blood flow of kidney 42L is functioning normally increases as the correlation coefficient approaches zero. Correlation threshold line 80 of the present disclosure is not limited to a value of 0.5, or to any particular value. The value of correlation threshold line 80 may be based on empirical data, and may vary depending on factors such as characteristics of patient 10, such as age, health, smoking habits, etc.

The lower limit of autoregulation (LLA) line marks a lower threshold for the MAP of patient 10 where normal autoregulation of the renal blood flow of kidney 42L occurs. Generally, when patient 10 has a MAP value that is above the LLA line, the autoregulation of the renal blood flow of kidney 42L is normal. When patient 10 has a MAP value below the LLA line, the autoregulation of the renal blood flow of kidney 42L is impaired. In the example of FIG. 9, the chart shows that the correlation coefficient between the changes of the flow rate of the renal blood flow of kidney 42L and the changes in the MAP of patient 10 is above correlation threshold line 80 when the MAP of patient 10 is below 50 mmHg. Thus, the LLA line for the renal blood flow of kidney 42L in the example of FIG. 9 is 50 mmHg. System processor 19 can set an alarm in blood flow monitor 12 such that blood flow monitor will alert medical personnel if the MAP of patient 10 approaches the LLA line or falls below the LLA line. If the alarm activates, medical personnel can be alerted that the MAP of patient 10 is below the LLA line and that the autoregulation of the renal blood flow of kidney 42L is likely impaired. Medical personnel can respond to the alarm by taking measures (such as administering fluids or medication to patient 10) to raise the MAP of patient 10 above the LLA line. Over time, as system processor 19 continues to monitor the MAP of patient 10, continues to monitor the changes in the flow rate of the renal blood flow, and continues to determine the real time value for the correlation coefficient and add that value to the chart of FIG. 9, the position of the LLA line may shift relative to the X-axis. System processor 19 can update the alarm of the blood flow monitor 12 to follow any changes in the LLA line. Blood flow monitor 12 can also include a hypotension prediction algorithm as part of AR monitoring module 32 and can use the LLA line as a threshold or definition for hypotension of patient 10.

FIG. 10 is a plot of the correlation coefficient between the changes in the flow rate of the renal blood flow of kidney 42L and the changes in the MAP of patient 10 versus the MAP of patient 10. The Y-axis represents the values of the correlation coefficient and the X-axis represents the values of the MAP of the patient 10 when the correlation coefficient was determined by system processor 19. Similar to the chart of FIG. 9, the plot of FIG. 10 shows the LLA line and correlation threshold line 80. The plot of FIG. 10 further includes the upper limit of autoregulation (ULA) line for the renal blood flow of kidney 42L, which is presently at about 105 mmHg. When the MAP of patient 10 is between the LLA line and the ULA line, the changes in the flow rate of the renal blood flow of kidney 42L do not correlate to the changes in the MAP of patient 10. Since the changes in the flow rate of the renal blood flow of kidney 42L do not correlate to the changes in the MAP of patient 10 when the MAP of patient 10 is between the LLA line and the ULA line, the autoregulation of the renal blood flow of kidney 42L functions normally when the MAP of patient 10 is between the LLA line and the ULA line. As discussed above with reference to FIG. 9, the autoregulation of the renal blood flow of kidney 42L is impaired when the MAP of patient 10 is below the LLA line. The autoregulation of the renal blood flow of kidney 42L is also impaired when the MAP of patient 10 is above the ULA line. When the MAP of patient 10 is above the ULA line, the autoregulation of the renal blood flow of kidney 42L becomes passive and changes in the flow rate of the renal blood flow of kidney 42L correlate with changes in the MAP of patient 10.

System processor 19 can set a first alarm in blood flow monitor 12 such that blood flow monitor 12 will alert medical personnel if the MAP of patient 10 approaches the LLA line or falls below the LLA line. System processor 19 can set a second alarm in blood flow monitor 12 if the MAP of patient 10 approaches the ULA line or exceeds the ULA line. If the first alarm activates, medical personnel can be alerted that the MAP of patient 10 is below the LLA line, or approaching the LLA line, and that the autoregulation of the renal blood flow of kidney 42L is likely impaired. Medical personnel can respond to the alarm by taking measures (such as administering fluids or medication to patient 10) to raise the MAP of patient 10 above the LLA line. If the second alarm activates, medical personnel can be alerted that the MAP of patient 10 is above the ULA line, or approaching the ULA line, and that the autoregulation of the renal blood flow of kidney 42L is likely impaired. Medical personnel can respond to the alarm by taking measures to lower the MAP of patient 10 below the ULA line.

System processor 19 can output the plot of FIG. 10 to display 28 and color code the plot to aid medical personnel in identifying when the autoregulation of the renal blood flow of kidney 42L is functional or impaired. For example, the regions of the plot of FIG. 10 above the ULA line and below the LLA line relative to the X-axis can be shaded red, while the region of the plot between the ULA line and the LLA line can be shaded green. Over time, as system processor 19 continues to monitor the MAP of patient 10, continues to monitor the changes in the flow rate of the renal blood flow, and continues to determine the real time value for the correlation coefficient and add that real-time value to the plot of FIG. 10, the position of the LLA line and/or the position of the ULA line may shift relative to the X-axis. System processor 19 can update the first alarm and the second alarm of the blood flow monitor 12 to follow any changes in the position of the LLA line or the ULA line.

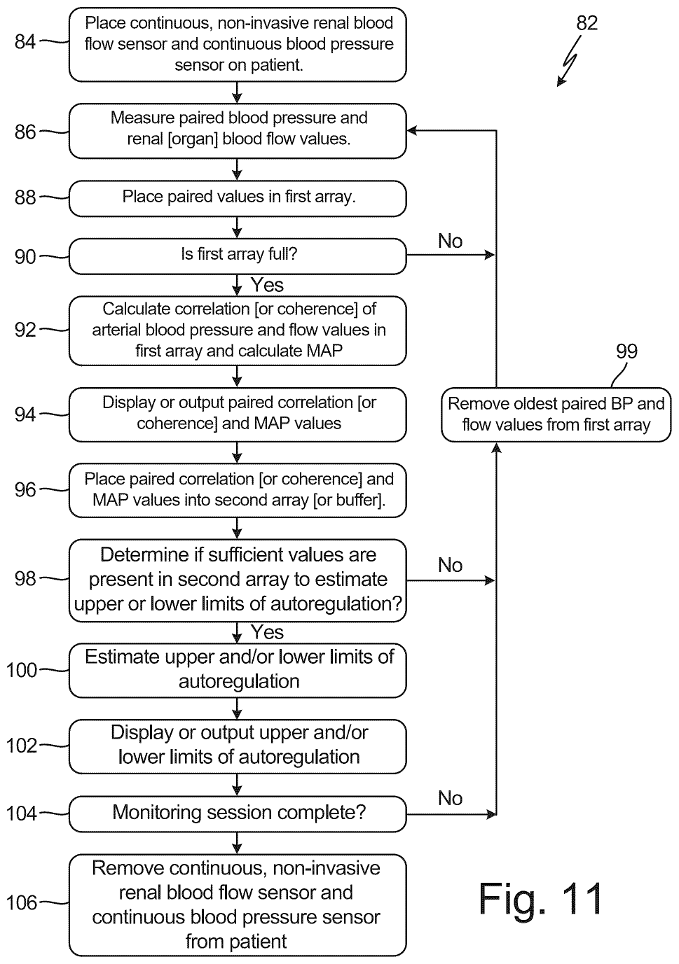

FIG. 11 is a block diagram of method 82 for determining the autoregulation profile of the renal blood flow of kidney 42L of patient 10. Medical personnel perform first step 84 of method 82 by placing non-invasive ultrasound transducer probe 14 and continuous hemodynamic pressure sensor 17 on patient 10. Second step 86 of method 82 is to measure paired values in time for the arterial pressure of patient 10 and the renal blood flow of kidney 42L using ultrasound transducer probe 14 and hemodynamic pressure sensor 17. System processor 19 performs third step 88 of method 82 by placing the paired values for the arterial pressure of patient 10 and the renal blood flow of kidney 42L into a first array. The first array is a first data buffer of system processor 19 and/or system memory 20 that stores the information for the paired values for the arterial pressure of patient 10 and the renal blood flow of kidney 42L for future processing by system processor 19.

Fourth step 90 of method 82 is an inquiry made by system processor 19. The inquiry of fourth step 90 is whether the first array is full. If the first array is not full, then system processor 19 returns to step 86 and repeats steps 86-90 until the first array is full. If the first array is full, system processor 19 proceeds with fifth step 92 of method 82. In fifth step 92 of method 82, system processor 19 calculates the mean arterial pressure (MAP) and the mathematical relationship between the arterial pressure values and the renal blood flow values in the first array. As discussed above, the calculated mathematical relationship between the arterial pressure values and the renal blood flow values can be the correlation (or coherence) between the change in the flow rate of the renal blood flow of kidney 42L and the change in the MAP of patient 10. In sixth step 94 of method 82, system processor 19 pairs the correlation (or coherence) values with the MAP values and outputs the paired correlation (or coherence) values and MAP values to display 28.

In seventh Step 96 of method 82, system processor 19 places the paired correlation (or coherence) values and the MAP values into a second array. The second array is a second data buffer of system processor 19 and/or system memory 20 that stores the information for the paired correlation (or coherence) values and the MAP values for future processing by system processor 19. System processor 19 performs eighth step 98 of method 82 by determining whether sufficient values are present in the second array to estimate the upper limit of autoregulation or the lower limit of autoregulation. If system processor 19 determines that the second array does not contain sufficient values to estimate the upper limit of autoregulation or the lower limit of autoregulation, system processor proceeds to ninth step 99 by removing the oldest pair of the paired values for the arterial pressure of patient 10 and the renal blood flow of kidney 42L in the first array. After removing the oldest pair of the paired values for the arterial pressure of patient 10 and the renal blood flow of kidney 42L in the first array, system processor proceeds with second step 86 and repeats steps 86-98. If system processor 19 determines that the second array is full, system processor proceeds with tenth step 100.

System processor 19 performs tenth step 100 of method 82 by estimating the upper limit of autoregulation and/or the lower limit of autoregulation. System processor 19 can fit the upper limit of autoregulation and/or the lower limit of autoregulation to a Lassen curve. In eleventh step 102 of method 82, system processor 19 can output the upper limit of autoregulation and/or the lower limit of autoregulation to display 28. Twelfth step 104 of method 82 is an inquiry for system processor 19. The twelfth step 104 inquires whether the monitoring session of kidney 42 of patient 10 is complete. If the answer is no, then system processor proceeds to ninth step 99 by removing the oldest pair of the paired values for the arterial pressure of patient 10 and the renal blood flow of kidney 42L in the first array. After removing the oldest pair of the paired values for the arterial pressure of patient 10 and the renal blood flow of kidney 42L in the first array, system processor proceeds with second step 86 and repeats steps 86-104. If system processor 19 determines that the monitoring session of kidney 42 is complete, then medical personnel will perform the thirteenth step 106 of method 82 by removing ultrasound transducer probe 14 and hemodynamic pressure sensor 17 from patient 10.

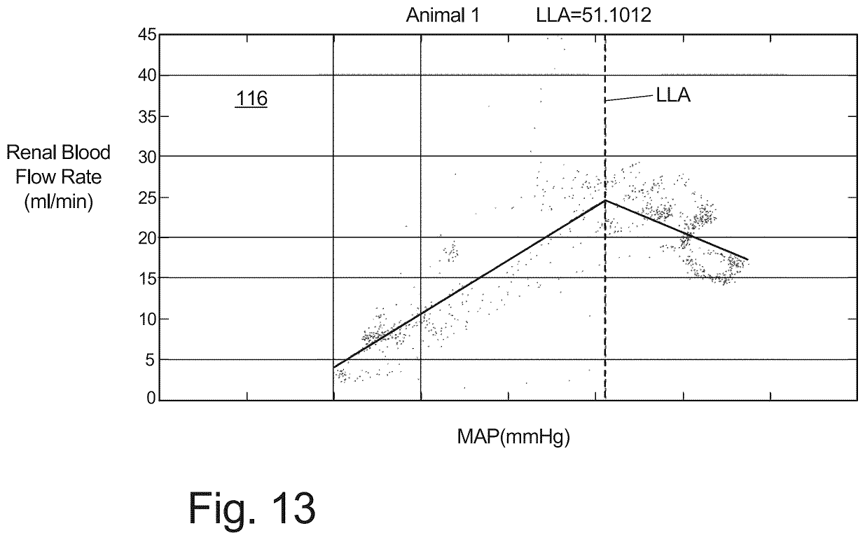

FIG. 12 is a chart from an experiment demonstrating monitoring system 11. The chart shows four plots. First plot 108 is a plot of mean arterial pressure (MAP) of a test subject (a pig) that was measured by hemodynamic pressure sensor 17 over time. First plot 108 also includes the lower limit of autoregulation (LLA) line of a renal blood flow of the test subject. The LLA line in first plot 108 is determined by plotting, as shown in plot 116 of FIG. 13, the renal blood flow of the test subject against the MAP of the test subject for each time sample and fitting two lines through the data points and assigning the inflection point between the two lines as the location of the LLA line. The LLA line marks a lower threshold for the MAP of the test subject where normal autoregulation of the renal blood flow of kidney 42L occurs. When the test subject has a MAP value that is above the LLA line, the autoregulation of the renal blood flow of kidney 42L is normal and correlation is low between the flow rate of the renal blood flow and the MAP of the test subject. When patient 10 has a MAP value below the LLA line, the autoregulation of the renal blood flow of kidney 42L is impaired and correlation is high (approaching 1) between the flow rate of the renal blood flow and the MAP of the test subject.

Second plot 110 is a plot of a flow rate of the renal blood flow of the test subject over time that was measured by an invasive flow probe that was surgically implanted around a renal artery of the test subject to provide a reference measurement of the renal blood flow. Third plot 112 is a plot of the flow rate of the renal blood flow of the test subject as measured by ultrasound transducer probe 14 of monitoring system 11 over time. Third plot 112 shows that non-invasive ultrasound transducer probe 14 of monitoring system 11 in this experiment was able to identify the changes in the renal blood flow of the test subject in a similar manner as the invasive transonic flow probe that was surgically implanted around the renal artery of the test subject.

Fourth plot 114 includes a first line representing the correlation between the changes in the MAP of the test subject and the changes in the flow rate of the renal blood flow of the test subject as measured by the invasive flow probe. Fourth plot 114 also includes a second line representing the correlation between the changes in the MAP of the test subject and the changes in the flow rate of the renal blood flow of the test subject as measured by ultrasound transducer probe 14 of monitoring system 11. A balloon catheter was inserted in the inferior vena cava of the test subject. During the experiment, the balloon catheter was inflated and deflated several times to cause decreases and increases in the MAP of the test subject.

When the balloon catheter was inflated, the MAP of the test subject would decrease below the LLA line, which caused the correlation lines of fourth plot 114 to increase and indicate a correlation between the change in the MAP and the changes in the flow rate of the renal blood flow of the test subject. The presence of the correlation between the change in the MAP and the changes in the flow rate of the renal blood flow of the test subject indicates that the autoregulation of the renal blood flow of the test subject is impaired. When the balloon catheter was deflated, the MAP of the test subject would increase above the LLA line, which caused the correlation lines of fourth plot 114 to decrease and indicate a non-correlation between the change in the MAP and the changes in the flow rate of the renal blood flow of the test subject. The presence of the non-correlation between the changes in the MAP and the changes in the flow rate of the renal blood flow of the test subject indicates that the autoregulation of the renal blood flow of the test subject is functional. As indicated in fourth plot 114, monitoring system 11 in this experiment was able to identify a correlation or a non-correlation between the changes in the MAP and the changes in the flow rate of the renal blood flow of the test subject, and determine whether the autoregulation of the renal blood flow of the subject was impaired or functional.

FIG. 13 shows plot 116 of the renal blood flow versus the MAP of the test subject from the experiment discussed with reference to FIG. 12. This is the physiological flow autoregulation functionality of the test subject. As shown in plot 116 of FIG. 13, when the MAP of the test subject is below the LLA line, the autoregulation of the renal blood flow of the test subject becomes impaired and passive such that the flow rate of the renal blood flow trends and follows changes in the MAP of the test subject. When the MAP of the test subject is above the LLA line, the autoregulation of the renal blood flow of the test subject is functional and the flow rate of the renal blood flow of the test subject no longer correlates with changes in the MAP of the test subject.

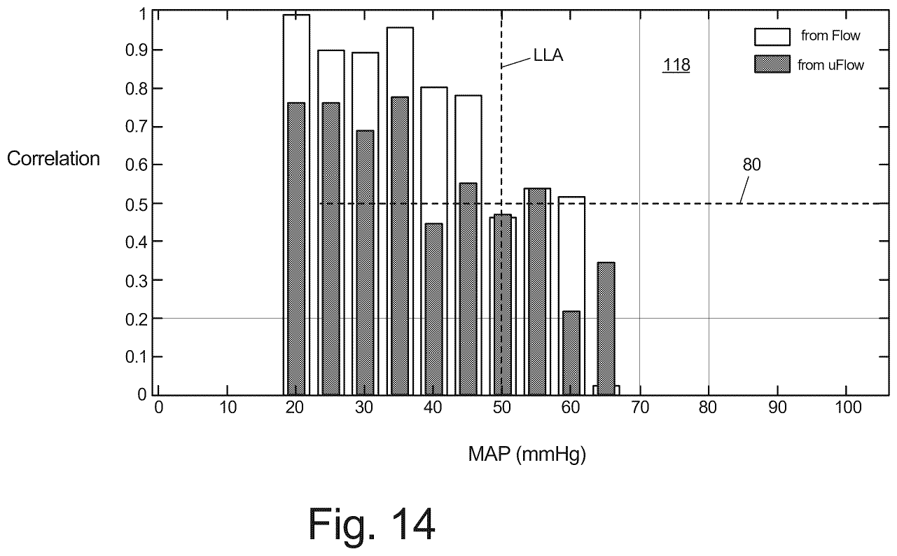

FIG. 14 shows chart 118 of correlation versus MAP of the test subject from the experiment of FIG. 12. Chart 118 of FIG. 14 is similar to the chart of FIG. 9. Chart 118 shows the correlation between the changes in the MAP of the test subject and the changes in the flow rate of the renal blood flow of the test subject versus the MAP of the test subject. Chart 118 compares the correlation that was determined by system processor 19 using renal blood flow rate data that was gathered by ultrasound transducer probe 14 and the correlation that was determined by system processor 19 using renal blood flow rate data that was gathered by the invasive flow probe that was surgically implanted around the renal artery of the test subject. Both sets of data show that the LLA line of the test subject is at about 50 mmHg. When the MAP of the test subject was below 50 mmHg, the autoregulation of the renal blood flow of the test subject was impaired and a correlation existed between the MAP of the test subject and the flow rate of the renal blood flow of the test subject. When the MAP of the test subject was above 50 mmHg, the autoregulation of the renal blood flow of the test subject was functional and no correlation existed between the MAP of the test subject and the flow rate of the renal blood flow of the test subject.

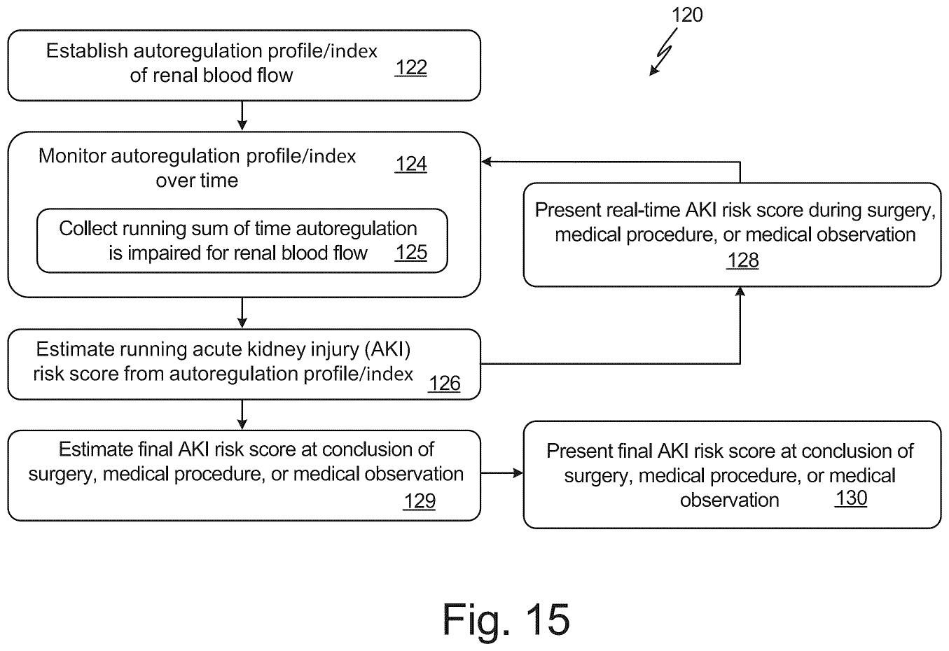

FIG. 15 is a block diagram of method 120 for operating monitoring system 11 shown in FIGS. 1-2 to continuously monitor the autoregulation of the renal blood flow of kidney 42L of patient 10 during a surgery, medical procedure, or medical observation. Autoregulation of the renal blood flow of kidney 42L is defined as the ability of the renal arteries and the renal veins to dilate and constrict in response to dynamic perfusion pressure changes to maintain the renal blood flow sufficient to the needs of kidney 42L. The changes in blood flow of kidney 42L are largely uncorrelated with changes in blood pressure of patient 10.

As discussed above with reference to FIGS. 2-14, monitoring system 11 uses a flow rate of the renal blood flow of kidney 42L estimated from the Doppler flow signal and the MAP of patient 10 to determine the autoregulation profile of kidney 42L of patient 10. System processor 19 monitors changes in the time domain and/or changes in the frequency domain for both the renal blood flow rate and the MAP of patient 10. System processor 19 evaluates relative to one another the changes in the renal blood flow rate and the changes in the MAP to determine the autoregulation profile of kidney 42L of patient 10. If system processor 19 determines a non-correlation between changes in the renal blood flow rate and changes in the MAP of patient 10, then system processor 19 determines that the autoregulation profile of kidney 42L is active and functioning properly. If system processor 19 determines a correlation exits between changes in the renal blood flow rate and changes in the MAP of patient 10, then system processor 19 determines that the autoregulation profile of kidney 42L is impaired. The Pearson correlation coefficient is an example of a time domain correlation that system processor 19 can use over a rolling time window to monitor the renal blood flow rate and the MAP of patient 10 for autoregulation. The Coherence Function, sometimes referred to as the Magnitude-Squared Coherence Function, is an example of a frequency domain correlation that system processor 19 can use to monitor the renal blood flow rate and the MAP of patient 10 for autoregulation.

Once ultrasound transducer probe 14 has been attached to patient 10 and is sensing the Doppler flow signal of the renal blood flow of kidney 42L and pressure sensor 70 has been attached to patient 10 and is sensing hemodynamic data representative of the MAP of patient 10, system processor 19 executes AR monitoring module 32 to perform first step 122 of method 120. In first step 122, system processor 19 executes AR monitoring module 32 to analyze the Doppler flow signal of the renal blood flow and the MAP of patient 10 to determine the autoregulation profile of the renal blood flow of kidney 42L and establish the LLA line and the ULA line of the autoregulation profile. Using the LLA and ULA lines, system processor 19 can determine when the autoregulation of the renal blood flow of kidney 42 is functional or impaired based on the value of the MAP of patient 10. Impaired autoregulation of the renal blood flow to kidney 42L over time can be indicative of injury to kidney 42L.

In second step 124 of method 120, system processor 19 executes AR monitoring module 32 to continuously monitor the Doppler flow signal of the renal blood flow for the autoregulation profile of the renal blood flow to kidney 42L during the surgery, medical procedure, or medical observation of patient 10. As part of second step 124, system processor 19 can output the autoregulation profile of the renal blood flow to display 28. In the example of FIG. 15, second step 124 of method 120 further includes sub-step 125. In sub-step 125, system processor 19 executes AR monitoring module 32 to collect a running sum of time that the autoregulation profile indicates that the autoregulation of the renal blood flow of kidney 42L is impaired during the surgery, the medical procedure, or the medical observation of patient 10.

In third step 126 of method 120, system processor 19 executes AR monitoring module 32 to estimate a real-time AKI risk score of patient 10 from the autoregulation profile of the renal blood flow. System processor 19 and AR monitoring module 32 use the running sum of the time that the autoregulation of the renal blood flow was impaired to estimate the real-time AKI risk score of patient 10. In fourth step 128 of method 120, system processor 19 outputs the real-time AKI risk score of patient 10 to display 28. The real-time AKI risk score can be shown on display 28 as a plot that shows how the real-time AKI risk score of patient 10 changes over time, and/or the real-time AKI risk score can be shown as a present value in injury score indicator 37. The real-time AKI risk score is recorded by system processor 19 into system memory 20. When estimating a next iteration of the real-time AKI risk score of patient 10, system processor 19 can use the recorded AKI risk score(s) in system memory 20 as part of the estimation of the next iteration of the real-time AKI risk score of patient 10. Thus, over time, the real-time AKI risk score of patient 10 is based on both real-time information from the autoregulation profile of the renal blood flow of kidney 42L plus cumulative past information of the autoregulation profile of the renal blood flow of kidney 42L.

As the surgery, the medical procedure, or the medical observation of patient 10 progresses, system processor 19 and AR monitoring module 32 continues to repeat second step 124, third step 126, and fourth step 128 of method 120 to continuously update and display the real-time AKI risk score of patient 10. Whenever monitoring system 11 indicates that the autoregulation of the renal blood flow of kidney 42L is impaired, monitoring system 11 can activate an alert or alarm to make medical personnel aware so that the medical personnel can take action to compensate for the impaired autoregulation or take action to restore autoregulation of the renal blood flow. At the end of the surgery, medical procedure, or medical observation of patient 10, system processor 19 can execute AR monitoring module 32 to perform fifth step 129 to estimate a final AKI risk score of kidney 42L of patient 10. System processor 19 can determine the final AKI risk score of patient 10 based on the values of the real-time AKI risk score that were tracked and recorded to system memory 20 throughout the surgery, medical procedure, or medical observation of patient 10. After estimating the final AKI risk score of kidney 42L, system processor 19 performs sixth step 130 of method 120 by outputting the final AKI risk score to display 28. Based on the value of the final AKI risk score, medical personnel can estimate if kidney 42L of patient 10 was injured during the surgery, medical procedure, or medical observation and can recommend that patient 10 seek treatment of kidney 42L.

The treatment techniques, methods, steps, etc. described or suggested herein or in references incorporated herein can be performed on a living animal or on a non-living simulation. Any of the various systems, devices, apparatuses, etc. in this disclosure can be sterilized (e.g., with heat, radiation, ethylene oxide, hydrogen peroxide, etc.) to ensure they are safe for use with patients, and the methods herein can comprise sterilization of the associated system, device, apparatus, etc. (e.g., with heat, radiation, ethylene oxide, hydrogen peroxide, etc.).

### DISCUSSION OF POSSIBLE EMBODIMENTS

The following are non-exclusive descriptions of possible embodiments of the present invention.

A method for continuously monitoring a kidney of a patient during a surgery, a medical procedure, or a medical observation includes continuously measuring a signal of a renal blood flow of the patient with a first sensor attached to the patient. The first sensor is in communication with a blood flow monitor. A processor of the blood flow monitor estimates a flow rate of the renal blood flow of the patient from the signal of the renal blood flow. The processor also monitors changes in the flow rate of the renal blood flow over time. An arterial pressure signal of the patient is continuously measured by a second sensor. The second sensor is in communication with the blood flow monitor. The processor also monitors changes in the arterial pressure signal over time and evaluates a mathematical relationship between the changes in the arterial pressure signal and the changes in the flow rate of the renal blood flow.

The method of the preceding paragraph can optionally include, additionally and/or alternatively, any one or more of the following features, configurations and/or additional components in the paragraphs below.

In an embodiment of the foregoing method, the method further comprises determining, by the processor, an autoregulation profile of the renal blood flow of the patient based on the mathematical relationship between the changes in the arterial pressure signal and the changes in the flow rate of the renal blood flow.

In an embodiment of the foregoing method, the method further comprises outputting in real time to a display in communication with the processor a representation of the flow rate of the renal blood flow over time, a representation of the arterial pressure signal over time, and/or a representation over time of the mathematical relationship between the changes in the arterial pressure signal and the changes in the flow rate of the renal blood flow.

In an embodiment of the foregoing method, the method further comprises continuously monitoring over time by the processor the mathematical relationship between the changes in the arterial pressure signal and the changes in the flow rate of the renal blood flow during the surgery, medical procedure, or medical observation of the patient.

In an embodiment of the foregoing method, the method further comprises evaluating by the processor a correlation coefficient representing correlation or non-correlation between the changes in the arterial pressure and the changes in the flow rate of the renal blood flow; wherein the processor determines the autoregulation profile of the renal blood flow of the patient based on the correlation coefficient.

In an embodiment of the foregoing method, the processor evaluates the correlation coefficient in a time domain.

In an embodiment of the foregoing method, the processor evaluates the correlation coefficient using a Pearson correlation coefficient computed over a rolling window of time.

In an embodiment of the foregoing method, the processor evaluates the correlation coefficient in a frequency domain.

In an embodiment of the foregoing method, the processor evaluates the correlation coefficient using a Coherence function computed across a prespecified frequency range.

In an embodiment of the foregoing method, the processor evaluates the correlation coefficient using a Coherence function computed from parameters of a transfer function of the arterial pressure signal and a transfer function of the flow rate of the renal blood flow.

In an embodiment of the foregoing method, the method further comprises setting, by the processor, a correlation threshold delineating a boundary above which the correlation coefficient represents correlation between the changes in the arterial pressure and the changes in the flow rate of the renal blood flow and below which the correlation coefficient represents non-correlation between the changes in the arterial pressure and the changes in the flow rate of the renal blood flow.

In an embodiment of the foregoing method, the method further comprises estimating, by the processor, a lower limit of autoregulation (LLA), wherein the LLA is an arterial pressure value of the patient below which the correlation coefficient is consistently above the correlation threshold.

In an embodiment of the foregoing method, the method further comprises estimating, by the processor, an upper limit of autoregulation (ULA), wherein the ULA is an arterial pressure value of the patient above which the correlation coefficient is consistently above the correlation threshold.

In an embodiment of the foregoing method, the method further comprises setting, by the processor, at least one alarm in the blood flow monitor that activates in response to the hemodynamic pressure sensor sensing an arterial pressure of the patient rising above the ULA or falling below the LLA.

In an embodiment of the foregoing method, the method further comprises estimating, by the processor of the blood flow monitor, a real-time acute kidney injury risk score of the patient from the autoregulation profile of the patient and a predetermined threshold; and outputting in real time to the display a representation of the real-time acute kidney injury risk score of the patient over time.

In an embodiment of the foregoing method, the predetermined threshold comprises at least one of the LLA and the ULA.

In an embodiment of the foregoing method, the method further comprises setting, by the processor, hypotension thresholds and/or definitions for a hypotension prediction algorithm of the blood flow monitor based on the autoregulation profile of the patient.

In an embodiment of the foregoing method, the hypotension thresholds and/or the definitions for the hypotension prediction algorithm of the blood flow monitor comprises at least one of the LLA and the ULA.

In an embodiment of the foregoing method, the method further comprises: collecting, by the blood flow monitor, a running sum of time that the changes in the arterial pressure signal and the changes in the flow rate of the renal blood flow correlate; estimating, by the processor of the blood flow monitor, a real-time acute kidney injury risk score of the patient from the running sum of time that the changes in the arterial pressure signal and the changes in the flow rate of the renal blood flow correlate; and outputting in real time to the display a representation of the real-time acute kidney injury risk score of the patient over time.

In an embodiment of the foregoing method, the method further comprises: continuously measuring the signal of the renal blood flow of the patient with the first sensor attached to the patient comprises: sampling a waveform of the signal of the renal blood flow at a rate of at least 10 Hz, at least 20 Hz, at least 60 Hz, at least 100 Hz, or at least 200 Hz; sampling the signal of the renal blood flow every cardiac cycle of the patient; sampling an average of the signal of the renal blood flow over a window of time; and/or sampling an average of the signal of the renal blood flow over a rolling window of time.

In an embodiment of the foregoing method, the signal of the renal blood flow is a relative change in the renal blood flow, a flow velocity of the renal blood flow, and/or a peak flow velocity of the renal blood flow.

In an embodiment of the foregoing method, the first sensor comprises an ultrasound transducer probe attached in a stationary position to an abdomen of the patient and the signal of the renal blood flow of the patient is a Doppler flow signal.

In an embodiment of the foregoing method, the method further comprises: positioning the ultrasound transducer probe on the abdomen of the patient and attaching the ultrasound transducer probe to the abdomen of the patient with an adhesive patch to maintain contact between the ultrasound transducer probe and the patient without an ultrasound operator; and scanning the abdomen of the patient with the ultrasound transducer probe to locate the Doppler flow signal of the renal blood flow of the patient.

In an embodiment of the foregoing method, the method further comprises executing beamformer software code by the processor to track-scan the Doppler flow signal of the renal blood flow of the patient with a two-dimensional phased array of transducer elements of the ultrasound transducer probe to continuously sense the Doppler flow signal of the renal blood flow of the patient during the surgery, medical procedure, or medical observation without an ultrasound operator.

In an embodiment of the foregoing method, the method further comprises: executing the beamformer software code by the processor to emit a set of sequential beams from the array of transducer elements to track a center of the renal blood flow relative to the array of transducer elements; focusing, by the processor executing the beamformer software code, each beam from the set of sequential beams in different locations; and adjusting, by the processor executing the beamformer software code, the position of the set of sequential beams onto the center of the renal blood flow to maintain the Doppler flow signal of the renal blood flow of the patient.

In an embodiment of the foregoing method, the second sensor comprises a hemodynamic pressure sensor attached to the patient by a radial arterial catheter.

In an embodiment of the foregoing method, the second sensor comprises a hemodynamic pressure sensor attached to the patient by a femoral arterial catheter.

In an embodiment of the foregoing method, the second sensor comprises a non-invasive hemodynamic pressure sensor.

In an embodiment of the foregoing method, the method further comprises setting, by the processor, blood pressure alarms in the blood flow monitor based on the autoregulation profile of the patient.

A system includes a first sensor configured to continuously measure a signal of a renal blood flow of a patient during a surgery, a medical procedure, or a medical observation. A second sensor is configured to continuously measure an arterial pressure signal of the patient during the surgery, the medical procedure, or the medical observation. A blood flow monitor is in communication with the first sensor and the second sensor. The blood flow monitor includes a system memory that stores monitoring software code and a processor. The processor is configured to execute the monitoring software code to estimate a flow rate of the renal blood flow of the patient from the signal of the renal blood flow and monitor changes in the flow rate of the renal blood flow over time. The processor is also configured to execute the monitoring software code to monitor changes in the arterial pressure signal over time and evaluate a mathematical relationship between the changes in the arterial pressure signal and the changes in the flow rate of the renal blood flow.

The system of the preceding paragraph can optionally include, additionally and/or alternatively, any one or more of the following features, configurations and/or additional components in the paragraphs below.

In an embodiment of the foregoing system, the processor is configured to execute the monitoring software code to: determine an autoregulation profile of the renal blood flow of the patient based on the mathematical relationship between the changes in the arterial pressure signal and the changes in the flow rate of the renal blood flow.

In an embodiment of the foregoing system, the signal of the renal blood flow comprises a Doppler flow signal and the first sensor comprises an ultrasound transducer probe comprising a two-dimensional array of transducer elements configured to continuously measure the Doppler flow signal of the renal blood flow of the patient during the surgery, the medical procedure, or the medical observation.

In an embodiment of the foregoing system, the two-dimensional array of transducer elements of the ultrasound transducer probe comprises a phased array of transducer elements.

In an embodiment of the foregoing system, the system memory stores probe control software code with beamformer software code, and wherein the processor is configured to execute the beamformer software code to: track-scan the Doppler flow signal of the renal blood flow of the patient by emitting multiple ultrasound beams from the phased array of transducer elements to track the Doppler flow signal of the renal blood flow of the patient relative to the phased array of transducer elements.

In an embodiment of the foregoing system, the second sensor comprises a hemodynamic pressure sensor connected to a radial arterial catheter.

In an embodiment of the foregoing system, the second sensor comprises a hemodynamic pressure sensor connected to a femoral arterial catheter.

In an embodiment of the foregoing system, the second sensor comprises a non-invasive hemodynamic pressure sensor.

In an embodiment of the foregoing system, the system further comprises a display in communication with the processor to receive and show a representation of the flow rate of the renal blood flow over time, a representation of the arterial pressure signal over time, and/or a representation over time of the mathematical relationship between the changes in the arterial pressure signal and the changes in the flow rate of the renal blood flow.

In an embodiment of the foregoing system, the processor is configured to execute the monitoring software code to: continuously monitor over time by the processor the autoregulation profile of the renal blood flow of the patient during the surgery, medical procedure, or medical observation of the patient.

In an embodiment of the foregoing system, the processor is configured to execute the monitoring software code to: evaluate a correlation or non-correlation between the changes in the arterial pressure and the changes in the flow rate of the renal blood flow; and determine the autoregulation profile of the renal blood flow of the patient based on the correlation or the non-correlation between the changes in the arterial pressure and the changes in the flow rate of the renal blood flow.

In an embodiment of the foregoing system, the processor is configured to execute the monitoring software code to: evaluate the correlation or the non-correlation between the changes in the arterial pressure and the changes in the flow rate of the renal blood flow in a time domain.

In an embodiment of the foregoing system, the processor is configured to execute the monitoring software code to: evaluate the correlation or the non-correlation between the changes in the arterial pressure and the changes in the flow rate of the renal blood flow using a Pearson correlation coefficient computed over a rolling window of time.

In an embodiment of the foregoing system, the processor is configured to execute the monitoring software code to: evaluate the correlation or the non-correlation between the changes in the arterial pressure and the changes in the flow rate of the renal blood flow in a frequency domain.

In an embodiment of the foregoing system, the processor is configured to execute the monitoring software code to: evaluate the correlation or the non-correlation between the changes in the arterial pressure and the changes in the flow rate of the renal blood flow using a Coherence function computed across a prespecified frequency range.

In an embodiment of the foregoing system, the processor is configured to execute the monitoring software code to: evaluate the correlation or the non-correlation between the changes in the arterial pressure and the changes in the flow rate of the renal blood flow using a Coherence function computed from parameters of a transfer function of the arterial pressure signal and a transfer function of the flow rate of the renal blood flow.

In an embodiment of the foregoing system, the processor is configured to execute the monitoring software code to: estimate a real-time acute kidney injury risk score of the patient from the autoregulation profile of the patient and a predetermined threshold; and output in real time to the display a representation of the real-time acute kidney injury risk score of the patient over time.

In an embodiment of the foregoing system, the processor is configured to execute the monitoring software code to: collect a running sum of time that the changes in the arterial pressure signal and the changes in the flow rate of the renal blood flow correlate; and estimate a real-time acute kidney injury risk score of the patient from the running sum of time that the changes in the arterial pressure signal and the changes in the flow rate of the renal blood flow correlate; and output in real time to the display a representation of the real-time acute kidney injury risk score of the patient over time.

In an embodiment of the foregoing system, the processor is configured to execute the monitoring software code to: set blood pressure alarms in the blood flow monitor based on the autoregulation profile of the patient.

In an embodiment of the foregoing system, the processor is configured to execute the monitoring software code to: set hypotension thresholds and/or definitions for a hypotension prediction algorithm of the blood flow monitor based on the autoregulation profile of the patient.

A method for continuously monitoring a kidney of a patient during a surgery, a medical procedure, or a medical observation, includes continuously measuring a Doppler flow signal of a renal blood flow of the patient with an ultrasound transducer probe. The ultrasound transducer probe is attached in a stationary position to an abdomen of the patient and is in communication with a processor of a blood flow monitor. The processor monitors changes in the renal blood flow over time. A hemodynamic pressure sensor continuously measures an arterial pressure signal of the patient. The hemodynamic pressure sensor is in communication with the blood flow monitor. The processor monitors changes in the arterial pressure signal over time and evaluates a mathematical relationship between the changes in the arterial pressure signal and the changes in the renal blood flow. The processor determines an autoregulation profile of the renal blood flow of the patient based on the mathematical relationship between the changes in the arterial pressure signal and the changes in the renal blood flow.

A method for continuously monitoring a kidney of a patient during a surgery, a medical procedure, or a medical observation, includes continuously measuring a Doppler flow signal of a renal blood flow of the patient with an ultrasound transducer probe. The ultrasound transducer probe is attached in a stationary position to an abdomen of the patient and is in communication with a processor of a blood flow monitor. The processor monitors changes in the renal blood flow over time. A hemodynamic pressure sensor continuously measures an arterial pressure signal of the patient. The hemodynamic pressure sensor is in communication with the blood flow monitor. The processor monitors changes in the arterial pressure signal over time and evaluates a mathematical relationship between the changes in the arterial pressure signal and the changes in the renal blood flow. The processor determines an autoregulation profile of the renal blood flow of the patient based on the mathematical relationship between the changes in the arterial pressure signal and the changes in the renal blood flow.

The method of the preceding paragraph can optionally include, additionally and/or alternatively, any one or more of the following features, configurations and/or additional components in the paragraphs below.

In an embodiment of the foregoing method, the processor is configured to execute the monitoring software code to: determine an autoregulation profile of the renal blood flow of the patient based on the mathematical relationship between the changes in the arterial pressure signal and the changes in the renal blood flow.

While the invention has been described with reference to an exemplary embodiment(s), it will be understood by those skilled in the art that various changes may be made and equivalents may be substituted for elements thereof without departing from the scope of the invention. In addition, many modifications may be made to adapt a particular situation or material to the teachings of the invention without departing from the essential scope thereof. Therefore, it is intended that the invention not be limited to the particular embodiment(s) disclosed, but that the invention will include all embodiments falling within the scope of the appended claims.

1. A method for continuously monitoring a kidney of a patient during a surgery, a medical procedure, or a medical observation, the method comprising:
continuously measuring a signal of a renal blood flow of the patient with a first sensor attached to the patient, wherein the first sensor is in communication with a blood flow monitor;
estimating, by a processor of the blood flow monitor, a flow rate of the renal blood flow of the patient from the signal of the renal blood flow;
monitoring, by the processor, changes in the flow rate of the renal blood flow over time;
continuously measuring an arterial pressure signal of the patient with a second sensor, wherein the second sensor is in communication with the blood flow monitor;
monitoring, by the processor, changes in the arterial pressure signal over time; and
evaluating, by the processor, a mathematical relationship between the changes in the arterial pressure signal and the changes in the flow rate of the renal blood flow.

2. The method of claim 1, further comprising:
determining, by the processor, an autoregulation profile of the renal blood flow of the patient based on the mathematical relationship between the changes in the arterial pressure signal and the changes in the flow rate of the renal blood flow.

3. The method of claim 1, further comprising:
outputting in real time to a display in communication with the processor a representation of the flow rate of the renal blood flow over time, a representation of the arterial pressure signal over time, and/or a representation over time of the mathematical relationship between the changes in the arterial pressure signal and the changes in the flow rate of the renal blood flow.

4. The method of claim 3, further comprising:
collecting, by the blood flow monitor, a running sum of time that the changes in the arterial pressure signal and the changes in the flow rate of a renal blood flow correlate;
estimating, by the processor of the blood flow monitor, a real-time acute kidney injury risk score of the patient from the running sum of time that the changes in the arterial pressure signal and the changes in the flow rate of the renal blood flow correlate; and
outputting in real time to the display a representation of the real-time acute kidney injury risk score of the patient over time.

5. The method of claim 1, further comprising:
continuously monitoring over time by the processor the mathematical relationship between the changes in the arterial pressure signal and the changes in the flow rate of the renal blood flow during the surgery, medical procedure, or medical observation of the patient.

6. The method of claim 1, further comprising:
evaluating by the processor a correlation coefficient representing correlation or non-correlation between the changes in the arterial pressure and the changes in the flow rate of the renal blood flow;
wherein the processor determines the autoregulation profile of the renal blood flow of the patient based on the correlation coefficient.

7. The method of claim 6, wherein the processor evaluates the correlation coefficient in a time domain, and wherein the processor evaluates the correlation coefficient using a Pearson correlation coefficient computed over a rolling window of time.

8. The method of claim 6, wherein the processor evaluates the correlation coefficient in a frequency domain, and wherein:
the processor evaluates the correlation coefficient using a Coherence function computed across a prespecified frequency range; or
the processor evaluates the correlation coefficient using a Coherence function computed from parameters of a transfer function of the arterial pressure signal and a transfer function of the flow rate of the renal blood flow.

9. The method of claim 6, further comprising:
setting, by the processor, a correlation threshold delineating a boundary above which the correlation coefficient represents correlation between the changes in the arterial pressure and the changes in the flow rate of the renal blood flow and below which the correlation coefficient represents non-correlation between the changes in the arterial pressure and the changes in the flow rate of the renal blood flow;
estimating, by the processor, a lower limit of autoregulation (LLA), wherein the LLA is an arterial pressure value of the patient below which the correlation coefficient is consistently above the correlation threshold;
estimating, by the processor, an upper limit of autoregulation (ULA), wherein the ULA is an arterial pressure value of the patient above which the correlation coefficient is consistently above the correlation threshold;
setting, by the processor, at least one alarm in the blood flow monitor that activates in response to the hemodynamic pressure sensor sensing an arterial pressure of the patient rising above the ULA or falling below the LLA;
estimating, by the processor of the blood flow monitor, a real-time acute kidney injury risk score of the patient from the autoregulation profile of the patient and a predetermined threshold; and
outputting in real time to the display a representation of the real-time acute kidney injury risk score of the patient over time;
wherein the predetermined threshold comprises at least one of the LLA and the ULA.

10. The method of claim 9, further comprising:
setting, by the processor, hypotension thresholds and/or definitions for a hypotension prediction algorithm of the blood flow monitor based on the autoregulation profile of the patient;
wherein the hypotension thresholds and/or the definitions for the hypotension prediction algorithm of the blood flow monitor comprises at least one of the LLA and the ULA.

11. The method of claim 1, wherein continuously measuring the signal of the renal blood flow of the patient with the first sensor attached to the patient comprises:
sampling a waveform of the signal of the renal blood flow at a rate of at least 10 Hz, at least 20 Hz, at least 60 Hz, at least 100 Hz, or at least 200 Hz;
sampling the signal of the renal blood flow every cardiac cycle of the patient;
sampling an average of the signal of the renal blood flow over a window of time; and/or
sampling an average of the signal of the renal blood flow over a rolling window of time.

12. The method of claim 1, wherein the signal of the renal blood flow is a relative change in the renal blood flow, a flow velocity of the renal blood flow, and/or a peak flow velocity of the renal blood flow.

13. The method of claim 1, wherein the first sensor comprises an ultrasound transducer probe attached in a stationary position to an abdomen of the patient and the signal of the renal blood flow of the patient is a Doppler flow signal, and further comprising:
positioning the ultrasound transducer probe on the abdomen of the patient and attaching the ultrasound transducer probe to the abdomen of the patient with an adhesive patch to maintain contact between the ultrasound transducer probe and the patient without an ultrasound operator;
scanning the abdomen of the patient with the ultrasound transducer probe to locate the Doppler flow signal of the renal blood flow of the patient;
executing beamformer software code by the processor to track-scan the Doppler flow signal of the renal blood flow of the patient with a two-dimensional phased array of transducer elements of the ultrasound transducer probe to continuously sense the Doppler flow signal of the renal blood flow of the patient during the surgery, medical procedure, or medical observation without an ultrasound operator;
executing the beamformer software code by the processor to emit a set of sequential beams from the array of transducer elements to track a center of the renal blood flow relative to the array of transducer elements;
focusing, by the processor executing the beamformer software code, each beam from the set of sequential beams in different locations; and
adjusting, by the processor executing the beamformer software code, the position of the set of sequential beams onto the center of the renal blood flow to maintain the Doppler flow signal of the renal blood flow of the patient.

14. The method of claim 1, wherein the second sensor comprises a hemodynamic pressure sensor attached to the patient by a radial arterial catheter or a femoral arterial catheter, or wherein the second sensor comprises a non-invasive hemodynamic pressure sensor.

15. The method of claim 1, further comprising:
setting, by the processor, blood pressure alarms in the blood flow monitor based on the autoregulation profile of the patient.

16. A system comprising:
a first sensor configured to continuously measure a signal of a renal blood flow of a patient during a surgery, a medical procedure, or a medical observation;
a second sensor configured to continuously measure an arterial pressure signal of the patient during the surgery, the medical procedure, or the medical observation;
a blood flow monitor in communication with the first sensor and the second sensor, wherein the blood flow monitor comprises:
a system memory that stores monitoring software code; and
a processor configured to execute the monitoring software code to:
estimate a flow rate of the renal blood flow of the patient from the signal of the renal blood flow;
monitor changes in the flow rate of the renal blood flow over time;
monitor changes in the arterial pressure signal over time; and
evaluate a mathematical relationship between the changes in the arterial pressure signal and the changes in the flow rate of the renal blood flow.

17. The system of claim 16, wherein the processor is configured to execute the monitoring software code to:
determine an autoregulation profile of the renal blood flow of the patient based on the mathematical relationship between the changes in the arterial pressure signal and the changes in the flow rate of the renal blood flow.

18. The system of claim 16, wherein the signal of the renal blood flow comprises a Doppler flow signal and the first sensor comprises an ultrasound transducer probe comprising a two-dimensional array of transducer elements configured to continuously measure the Doppler flow signal of the renal blood flow of the patient during the surgery, the medical procedure, or the medical observation.

19. The system of claim 18, wherein the two-dimensional array of transducer elements of the ultrasound transducer probe comprises a phased array of transducer elements, wherein the system memory stores probe control software code with beamformer software code, and wherein the processor is configured to execute the beamformer software code to:
track-scan the Doppler flow signal of the renal blood flow of the patient by emitting multiple ultrasound beams from the phased array of transducer elements to track the Doppler flow signal of the renal blood flow of the patient relative to the phased array of transducer elements.

20. The system of claim 16, wherein the second sensor comprises a hemodynamic pressure sensor connected to a radial arterial catheter or a femoral arterial catheter, or wherein the second sensor comprises a non-invasive hemodynamic pressure sensor.

21. The system of claim 16, further comprising:
a display in communication with the processor to receive and show a representation of the flow rate of the renal blood flow over time, a representation of the arterial pressure signal over time, and/or a representation over time of the mathematical relationship between the changes in the arterial pressure signal and the changes in the flow rate of the renal blood flow.

22. The system of claim 21, wherein the processor is configured to execute the monitoring software code to:
estimate a real-time acute kidney injury risk score of the patient from the autoregulation profile of the patient and a predetermined threshold; and
output in real time to the display a representation of the real-time acute kidney injury risk score of the patient over time.

23. The system of claim 21, wherein the processor is configured to execute the monitoring software code to:
collect a running sum of time that the changes in the arterial pressure signal and the changes in the flow rate of the renal blood flow correlate; and
estimate a real-time acute kidney injury risk score of the patient from the running sum of time that the changes in the arterial pressure signal and the changes in the flow rate of the renal blood flow correlate; and
output in real time to the display a representation of the real-time acute kidney injury risk score of the patient over time.

24. The system of claim 16, wherein the processor is configured to execute the monitoring software code to:
evaluate a correlation or non-correlation between the changes in the arterial pressure and the changes in the flow rate of the renal blood flow; and
determine the autoregulation profile of the renal blood flow of the patient based on the correlation or the non-correlation between the changes in the arterial pressure and the changes in the flow rate of the renal blood flow.

25. The system of claim 16, wherein the processor is configured to execute the monitoring software code to:
set blood pressure alarms in the blood flow monitor based on the autoregulation profile of the patient; and/or
set hypotension thresholds and/or definitions for a hypotension prediction algorithm of the blood flow monitor based on the autoregulation profile of the patient.
